(19) United States
(12) Patent Application Publication
(10) Pub. No.: US 2025/0215057 A1
(43) Pub. Date: Jul. 3, 2025

(54) Method for Preparing Outer Membrane Vesicles

(71) Applicant: GLAXOSMITHKLINE BIOLOGICALS SA, Rixensart (BE)

(72) Inventors: Francesca Micoli, Siena (IT); Gianmarco Gasperini, Siena (IT); Francesco Citiulo, Siena (IT); Laura Bartle Martin, Montgomery Village, MD (US); Allan James Saul, Belconnen, Australian Capital Territory (AU)

(21) Appl. No.: 18/721,985

(22) PCT Filed: Dec. 22, 2022
(86) PCT No.: PCT/EP2022/087522

§ 371 Date: Jun. 20, 2024

(30) Foreign Application Priority Data
Dec. 23, 2021 (GB) 2118913.9

(51) Int. Cl. C07K 14/25; A61K 39/00; A61P 31/04; C12N 1/20; C12N 9/10

(52) U.S. Cl. CPC C07K 14/25; A61P 31/04; C12N 1/20; C12N 9/1025; C12N 9/1048; A61K 39/00; A61K 2039/522; A61K 2039/523; A61K 2039/55505; A61K 2039/70

(57) ABSTRACT

The present invention relates to Shigella outer membrane vesicles comprising at least one heterologous O-antigen, a recombinant Shigella bacterium comprising at least one heterologous O-antigen, methods for preparing the outer membrane vesicles or recombinant Shigella bacterium, plasmids for use in the methods, immunogenic compositions, vaccines and methods of treatment.

### FIELD OF THE INVENTION

The present invention relates to Shigella outer membrane vesicles comprising at least one heterologous O-antigen, a recombinant Shigella bacterium comprising at least one heterologous O-antigen, methods for preparing the outer membrane vesicles or recombinant Shigella bacterium, plasmids for use in the methods, immunogenic compositions, vaccines and methods of treatment.

### BACKGROUND TO THE INVENTION

Gram-negative bacteria spontaneously release exosomes from their outer membrane, also called Outer Membrane Vesicles (OMV), containing surface exposed antigens in their native environment together with immuno-stimulatory molecules, such as lipopolysaccharide (LPS), lipoproteins and peptidoglycans. GMMA are OMV derived from bacteria genetically engineered to enhance the natural vesiculation. Recently GMMA have been proposed as an innovative vaccine platform for the delivery of the O-Antigen (OAg), known to be the key target for protective immunity against several pathogens.

Shigella is a major cause of disease with >200,000 deaths annually. Almost all deaths occur in developing countries and many of them in children under the age of five years. Protective immunity following Shigella infection seems to be predominantly directed against the serotype specific OAg and many OAg-based vaccines are currently under development. The Shigella genus is divided into 4 species and more than 50 antigenically distinct serotypes. The prevalence of these serotypes varies by country economic status, between geographical regions and changes over time even within the same region. S. sonnei is the most geographically widespread and dominates in economically developed countries, S. flexneri serotypes are more important in developing countries while S. boydii and S. dysenteriae occur at much lower frequencies. All these factors contribute to the complexity of vaccine development which needs to compromise between the number of components resulting in sufficient coverage and manufacturing affordability for Low- and Middle-Income Countries (LMIC). Two recent prospective studies examined Shigella diversity to inform vaccine development: the Multicenter Shigella Surveillance (MCSS) study and the Global Enteric Multicenter (GEM) study. Both studies highlighted the need to include S. sonnei and different S. flexneri serotypes in a broadly-protective vaccine.

The development of Shigella vaccines has focused on creating vaccines containing O-antigen from the most prevalent and/or severe Shigella strains. However, there is a concern that the introduction of vaccines against a small number of Shigella strains may induce serotype replacement, where the prevalence of the strains targeted by the vaccine decreases, but prevalence of other serotypes increases.

Thus, vaccines and methods of preparing vaccines against a higher number of Shigella strains and/or methods of preparing vaccines against a particular strain of Shigella quickly would be clearly advantageous.

### SUMMARY OF THE INVENTION

Different Shigella serotypes, such as different S. flexneri serotypes are distinguished in that they comprise different characteristic O-antigen having different glucosylation and O-acetylation patterns. These glucosylation and O-acetylation patterns are created by enzymes that modify O-antigen. For example, enzymes GtrI, GtrII, GtrIV, GrtrV, GtrX and OacA are involved in establishing the glucosylation and acetylation patterns on S. flexneri O-antigen.

The present application demonstrates that providing complementing plasmids comprising genes encoding one or more of enzymes that modify a Shigella O-antigen can be used to grow Shigella bacteria comprising O-antigen having modified glucosylation and acetylation patterns. In particular, the Shigella bacteria may comprise a mixture of different O-antigen or hybrid O-antigen. Outer membrane vesicles prepared from such Shigella may comprise O-antigen comprising epitopes from multiple serotypes of Shigella and are useful in vaccines as they can readily protect against multiple serotypes of Shigella.

Furthermore, the methods of the invention are advantageous as they enable the user to quickly produce vaccines against a range of different Shigella bacteria. The methods of the invention use plasmids comprising genes encoding enzymes that modify Shigella O-antigen, such as gtrI, gtrII, gtrIV, grtrV, gtrX and oacA, to modify the O-antigen of Shigella bacteria. The modified recombinant Shigella bacteria will comprise the modified O-antigen in the outer membrane, meaning that vaccines comprising the Shigella bacteria may be produced that protect against Shigella serotypes having O-antigen similar to the modified O-antigen. Similarly, the Shigella bacteria that comprise the modified O-antigen in the outer membrane may be used to produce outer membrane vesicles comprising the modified O-antigen that may also be used in vaccines. Such methods are advantageous, as they allow the user to take a well-characterized and safe strain of Shigella, and use that strain to produce vaccines that can protect against a range of different Shigella serotypes more quickly and easily that using prior art methods.

Thus, in a first aspect, the invention provides an outer membrane vesicle comprising at least one heterologous O-antigen.

In a second aspect, the invention provides a recombinant Shigella bacterium comprising at least one heterologous O-antigen.

In a third aspect, the invention provides a method for preparing Shigella outer membrane vesicles comprising:
(i) providing at least one gene encoding an enzyme that modifies a Shigella O-antigen on at least one plasmid;
(ii) culturing a production Shigella bacterium in the presence of the at least one plasmid under conditions suitable for the enzyme to be expressed by the production Shigella bacterium and for Shigella outer membrane vesicles to be produced; and
(iii) harvesting the outer membrane vesicles.

In a fourth aspect, the invention provides a method for preparing a recombinant Shigella bacterium comprising at least one heterologous O-antigen comprising:
(i) providing at least one gene encoding an enzyme that modifies a Shigella O-antigen on at least one plasmid;
(ii) culturing a production Shigella bacterium in the presence of the at least one plasmid under conditions suitable for the enzyme to be expressed by the production Shigella bacterium to produce the recombinant Shigella bacterium comprising at least one heterologous O-antigen; and
(iii) harvesting the recombinant Shigella bacterium comprising at least one heterologous O-antigen.

In a fifth aspect, the invention provides a plasmid comprising at least one gene encoding an enzyme that modifies a Shigella O-antigen.

In a sixth aspect, the invention provides an outer membrane vesicle or Shigella bacterium obtained by the method of the invention.

In a seventh aspect, the invention provides an outer membrane vesicle or Shigella bacterium obtainable by the method of the invention.

In an eighth aspect, the invention provides an immunogenic composition comprising the outer membrane vesicle or recombinant Shigella bacterium of the invention.

In a ninth aspect, the invention provides a vaccine comprising the immunogenic composition of the invention.

In a tenth aspect, the invention provides a method for preventing or treating Shigella infection, comprising administering an effective amount of the immunogenic composition or vaccine of the invention to a subject.

In an eleventh aspect, the invention provides a use of the immunogenic composition or the vaccine of the invention in the manufacture of a medicament for use in a method of preventing or treating Shigella infection.

### BRIEF DESCRIPTION OF THE FIGURES

FIG. 1—Schematic showing the strategy for generating the S. flexneri serotype Y scaffold strain, the complementation strategy used to convert the S. flexneri serotype Y scaffold strain to different S. flexneri serotypes, and the resulting OAg (O-Antigen) structures of the different S. flexneri serotypes as verified by ¹H-NMR. The numbers inside the circles show the position of the linkage of glucose to the RU.

FIG. 2—Schematic showing the OAg structures of strains generated by complementing the S. flexneri serotype Y scaffold strain with non-naturally-occurring combinations of OAg-modifying enzymes as verified by ¹H-NMR.

FIG. 3—¹H-NMR spectra of OAg isolated from GMMA of the converted scaffold strains and of the corresponding natural strains. Chemical shifts were assigned in the anomeric and O-acetyl regions. (A) compares the OAg isolated from GMMA of a 1a strain produced using the methods of the invention with the OAg isolated from GMMA of the natural 1a strain; (B) compares the OAg isolated from GMMA of a 2a strain produced using the methods of the invention with the OAg isolated from GMMA of the natural 2a strain. (i)-(iv) describe the predicted chemical structures of the OAg.

FIG. 4—Bactericidal activity of sera from mice immunized with GMMA of strains produced using methods of the invention (labelled GMMA 1a scaffold and GMMA 2a scaffold) and of the corresponding natural strains. Serum dilutions capable of killing 50% of bacteria in the assay (IC50) are reported for individual mice. (A) compares bactericidal activities against the serotype 1a strain; (B) compares bactericidal activities against the serotype 2a strain.

FIG. 5—Anomeric region of ¹H-NMR spectra of OAg isolated from GMMA comprising a mixture (mixed) or hybrid OAg and from GMMA of the corresponding individual native serotypes. (A) compares the OAg isolated from GMMA displaying mixed (1+2) OAg with the OAg isolated from GMMA of the serotype 1a strain and the OAg isolated from GMMA of the serotype 2a strain; (B) compares the OAg isolated from GMMA displaying hybrid (1+3) OAg with the OAg isolated from GMMA of the serotype 1a strain and the OAg isolated from GMMA of the serotype 3a strain.

FIG. 6—¹H-NMR spectra showing the lot-to-lot consistency in structural features of three different independently grown GMMA preparations. Bottom lines correspond to replicate 1; middle lines correspond to replicate 2; top lines correspond to replicate 3. (A) shows the consistency of preparations of GMMA displaying mixed (1+2) OAg—replicate 1 had a Rha/GLC molar ratio (HPAEC-PAD) of 2.4, replicate 2 had a Rha/GLC molar ratio (HPAEC-PAD) of 2.5, and replicate 3 had a Rha/GLC molar ratio (HPAEC-PAD) of 2.8; (B) shows the consistency of preparations of GMMA displaying hybrid (1+3) OAg—replicate 1 had a Rha/GLC molar ratio (HPAEC-PAD) of 1.5, replicate 2 had a Rha/GLC molar ratio (HPAEC-PAD) of 1.5, and replicate 3 had a Rha/GLC molar ratio (HPAEC-PAD) of 1.5.

FIG. 7—Bactericidal activity of sera from mice immunized with GMMA displaying mixed or hybrid OAg and from mice immunized with physical mixtures of GMMA displaying the corresponding individual serotypes. Serum dilutions capable of killing 50% of bacteria in the assay (IC50) are reported for individual mice. (A) compares GMMA displaying mixed (1+2) serotypes with physical mixtures of GMMA of the serotype 1a strain and GMMA of the serotype 2a strain; (B) compares GMMA displaying hybrid (1+3) serotypes with physical mixtures of GMMA of the serotype 1a strain and GMMA of the serotype 3a strain.

FIG. 8—Bactericidal activity of sera from mice immunized with GMMA displaying hybrid (1+3) OAg against a panel of the most epidemiologically relevant S. flexneri serotypes.

FIG. 9—Sequence Listing

### GENERAL DEFINITIONS

Unless defined otherwise, technical and scientific terms used herein have the same meaning as commonly understood by a person skilled in the art to which this invention belongs.

In general, the term “comprising” is intended to mean including but not limited to. For example, the phrase “An outer membrane vesicle comprising at least one heterologous antigen” should be interpreted to mean that the outer membrane vesicle comprises at least one heterologous antigen, but the outer membrane vesicle may comprise further components.

In some embodiments of the invention, the word “comprising” is replaced with the phrase “consisting of”. The term “consisting of” is intended to be limiting. For example, the phrase “An outer membrane vesicle consisting of at least one heterologous antigen” should be understood to mean that the outer membrane vesicle has at least one heterologous antigen and no further components.

In some embodiments of the invention, the word “comprising” is replaced with the phrase “consisting essentially of”. The term “consisting essentially of” means that specific further components can be present, namely those not materially affecting the essential characteristics of the subject matter.

The term “about” or “around” when referring to a value refers to that value but within a reasonable degree of scientific error. Optionally, a value is “about x” or “around x” if it is within 10%, within 5%, or within 1% of x.

The singular forms “a”, “an”, and “the” include plural referents unless the content clearly dictates otherwise. Thus, for example, reference to “the GMMA” includes two or more instances or versions of such GMMA.

All publications, patents and patent applications cited herein, whether supra or infra, are hereby incorporated by reference in their entirety.

### An Outer Membrane Vesicle

In an aspect, the invention relates to an outer membrane vesicle comprising at least one heterologous O-antigen.

An outer membrane vesicle is a vesicle formed from the outer membrane of a Gram negative bacterium. The outer membrane vesicles of the invention are formed from the outer membrane of a Shigella bacterium. The outer membrane vesicle may be any suitable outer membrane vesicle. In an embodiment, the outer membrane vesicle are GMMA, native outer membrane vesicles or detergent-extracted outer membrane vesicles. Native outer membrane vesicles are outer membrane vesicles which are released spontaneously from Gram-negative bacteria. Detergent-extracted outer membrane vesicles are outer membrane vesicles extracted from the outer membrane of Gram-negative bacteria using at least one detergent.

Generalised Modules for Membrane Antigens or GMMA are particles derived from the outer membrane of Gram-negative bacteria that have high levels of LPS, lipoproteins, proteins and other antigens that activate the innate immune response. GMMA are produced from genetically-modified bacterial strains that are mutated to enhance vesicle production and to remove or modify antigens (e.g. lipid A). Enhanced spontaneous generation of vesicles can be achieved, for example, by targeted deletion of proteins involved in maintenance of membrane integrity (see below). The outer surface of GMMA corresponds to the outer surface of the bacterium from which they are derived, preserving all membrane antigens (including e.g. lipopolysaccharides, lipooligosaccharides, lipoproteins, proteins) in the context of the membrane. GMMA (unlike detergent-extracted OMVs) retain these outer membrane components in their native conformation and correct orientation, better preserving immunogenicity against the bacterial strain from which they are derived. Thus, GMMA are highly immunogenic.

S. flexneri GMMA of the invention typically have a diameter of from 25 nm to 140 nm by electron microscopy, for example from 25 nm to 40 nm. GMMA may also have a bimodal size distribution. For example, the majority of GMMA having an average size from 25 nm to 40 nm in diameter (by EM) and a fraction of the particles having an average size from 65 nm to 140 nm. Particularly, at least 70%, at least 71%, at least 72%, at least 73%, at least 74%, at least 75%, at least 80%, at least 85%, or at least 90% of the GMMA will have a diameter of from 25 nm to 140 nm.

Outer membrane vesicles containing immunogenic compositions of the invention may be substantially free from whole bacteria, whether living or dead. The size of GMMA means that they can readily be separated from whole bacteria by filtration e.g. as typically used for filter sterilisation. Although GMMA will pass through standard 0.22 μm filters, these can rapidly become clogged by other material, and so it may be useful to perform sequential steps of filter sterilisation through a series of filters of decreasing pore size before using a 0.22 μm filter. Examples of preceding filters would be those with a pore size of 0.8 μm, 0.45 μm, etc. GMMA are spontaneously-released from bacteria and separation from the culture medium, for example, using filtration, is convenient. Optionally, the outer membrane vesicles are not formed by methods which involve deliberate disruption of the outer membrane (e.g. by detergent treatment, such as deoxycholate-extraction, or sonication) to cause outer membrane vesicles to form. GMMA of the invention are substantially free from inner membrane and cytoplasmic contamination and contain lipids and proteins.

The outer membrane vesicles of the present invention may be produced from a recombinant Shigella bacterium of the invention. The outer membrane vesicles of the present invention may be obtained by or obtainable by a method of the invention.

### A Recombinant Shigella Bacterium

In an aspect, the invention relates to a recombinant Shigella bacterium. Shigella are Gram-negative non-motible facultative anaerobic bacilli that fall into four serogroups S. dysenteriae, S. flexneri, S. boydii and S. sonnei. S. flexneri, S. sonnei, S. boydii, and S. dysenteriae express O-antigen in the outer membrane.

In a preferred embodiment, the recombinant Shigella bacterium is an S. flexneri bacterium. There are several different serotypes of S. flexneri distinguished by the nature of the O-antigen expressed by the bacterium. All serotypes of S. flexneri, except serotype 6, share a conserved polysaccharide backbone (corresponding to serotype Y) consisting of the following OAg repeating unit (RU): →2)-α-L-RhapIII-(1→2)-α-L-RhapII-(1→3)-α-L-RhapI-(1→3)-β-D-GlcpNAc-(1→.

The diversity of S. flexneri serotypes is due to the modification of this common OAg backbone with glucosyl and/or O-Acetyl groups, as a result of bacteriophage infection and acquisition of OAg-modifying enzymes.

The Shigella bacterium of the invention is recombinant, meaning that it has been engineered to comprise genetic material not present in a Shigella bacterium. Optionally, the Shigella bacterium of the invention is modified to comprise modified lipid A, as described in more detail under the heading “Detoxification”. Optionally, the Shigella bacterium of the invention is modified to comprise at least one hyber-blebbing mutation as discussed under the heading “Hyper blebbing”.

The terms “recombinant Shigella bacteria” or “a production Shigella bacterium” should be considered herein to refer to a single Shigella bacterium, but also a population of Shigella bacteria. For example, methods of the invention use a “production Shigella bacterium” and in reality a population of Shigella bacteria is used. Optionally, the population of Shigella bacteria used is sufficiently large to allow industrial scale production of recombinant Shigella bacteria and/or outer membrane vesicles.

### An Outer Membrane Vesicle or a Shigella Bacterium Comprising a Heterologous O-Antigen

As noted above, Shigella bacteria express O-antigen (O—Ag) in their outer membrane and the nature of the O-antigen determines, for example, the serotype of the Shigella bacterium. The present invention relates, in one aspect, to a Shigella bacterium comprising a heterologous O-antigen. In such aspects, the Shigella bacterium has been engineered to express an O-antigen that it would not naturally express. For example, a serotype 2a S. flexneri bacterium could be engineered to express an O-antigen from serotype 1a.

It is within the abilities of the skilled person to determine whether a recombinant Shigella bacterium comprises at least one heterologous O-antigen. A recombinant Shigella bacterium will be derived from a parent (non-engineered strain). The skilled person may determine the serotype of the parent (non-engineered strain) by determining the identity of the O-antigen it expresses using one of the techniques discussed in Example 1 under the heading “OAg purification and characterization”. For example, the skilled person may determine the identity of the OAg by using a combination of HPAEC-PAD (to determine sugar content) and GLC-MS to determine the linkage positions for the consistent sugars. Alternatively, the skilled person could use ¹H-NMR to determine the O-antigen identity. The skilled person may then determine whether the O-antigen of the recombinant Shigella bacteria is different by performing the same test for O-Antigen identity as performed on the parent strain, comparing the O-antigen identity of the parent and recombinant strains and determining whether they are different. If the parent strain O-antigen identity is different compared to the recombinant Shigella O-antigen identity, then the recombinant Shigella comprises a heterologous O-antigen. Notably, and as will be described in more detail below, it is possible for Shigella bacteria prepared using the methods of the invention to comprise O-antigen comprising epitopes from more than one Shigella serotype. In such cases, the Shigella bacteria will express at least one heterologous O-antigen, as Shigella bacteria only express one species of O-antigen.

Alternatively, the recombinant Shigella bacterium (such as recombinant Shigella bacteria produced using the methods of the invention) may comprise a plasmid comprising at least one gene encoding an enzyme that modifies a Shigella O-antigen on at least one plasmid. In such cases, it is possible to determine whether the recombinant Shigella bacterium comprise at least one heterologous O-antigen by determining whether the recombinant Shigella bacterium comprises at least one gene encoding an enzyme that modifies a Shigella O-antigen. One can make this determination by amplifying non-genomic DNA extracted from the recombinant Shigella bacterium, and sequencing the genetic material.

The present invention also relates to an outer membrane vesicle comprising at least one heterologous O-antigen. An outer membrane vesicle comprises at least one heterologous O-antigen if it is produced by a Shigella bacterium that comprises at least one heterologous O-antigen. Thus, it is possible to determine whether an outer membrane vesicle comprises at least one heterologous O-antigen by determining whether the Shigella bacterium from which the outer membrane vesicle is derived comprises a heterologous O-antigen (as described in the preceding paragraphs).

O-Antigen Comprising Epitopes from at Least Two Serotypes of Shigella

In some embodiments, the heterologous O-antigen comprises O-antigen comprising epitopes from at least two different serotypes of Shigella, for example at least two different serotypes of S. flexneri. Optionally, the outer membrane vesicle or recombinant Shigella bacterium of the invention comprises at least one heterologous O-antigen and the at least one heterologous O-antigen comprises epitopes from at least two different serotypes of Shigella, i.e. the outer membrane vesicle or recombinant Shigella bacterium of the invention comprises O-antigen that comprises epitopes from at least two different serotypes of Shigella.

For the purposes of the present invention the terms “serotype” and “serogroup” are used synonymously and S. sonnei strains may be considered to be a different serotype compared to any serotype of S. flexneri.

Optionally, the heterologous O-antigen comprises O-antigen comprising epitopes from at least two different serotypes of S. flexneri. The O-antigen epitopes of a number of Shigella flexneri strains are known, and these are set out in the following table:

TABLE 1
List of all the S. flexneri OAg-modifying enzymes

| | Resulting OAg | Conferred serotype | Epitope of |
|---|---|---|---|
| Gene | modification | specificity | serotype: |
| gtrI | α(1-4) glucosylation of | Type I | 1a |
| | GlcNAc | | |
| gtrII | α(1-4) glucosylation of | Type II | 2a |
| | RhaI | | |
| gtrIV | α(1-6) glucosylation of | Type IV | 4a |
| | GlcNAc | | |
| gtrV | α(1-3) glucosylation of | Type V | 5a |
| | RhaII | | |
| gtrX | α(1-4) glucosylation of | Group 7(8) | x |
| | RhaIII | | |
| oacA | 2-O-Acetylation of RhaI | Group 6 | 3b |
| oacB | 3-O-Acetylation of RhaIII | Group 9 | N/A |
| oacD | 6-O-Acetylation of | Group 10 | N/A |
| | GlcNAc | | |

As can be seen from this table, α(1-4) glucosylation of GlcNAc is the O-antigen epitope from serotype 1a, α(1-4) glucosylation of RhaI is the O-antigen epitope from serotype 2a, α(1-6) glucosylation of GlcNAc is the O-antigen epitope from serotype 4a, α(1-3) glucosylation of RhaII is the O-antigen epitope from serotype 5a, α(1-4) glucosylation of RhaIII is the O-antigen epitope from serotype x and 2-O-Acetylation of RhaI is the O-antigen epitope from serotype 3b. Thus, if a Shigella bacterium or an outer membrane vesicle comprises O-antigen comprising α(1-4) glucosylation of GlcNAc and α(1-4) glucosylation of RhaI, the Shigella bacterium or outer membrane vesicle will be considered to comprise O-antigen comprising epitopes from two different serotypes if S. flexneri (specifically serotypes 1a and 2a).

In such embodiments, the outer membrane vesicle or the recombinant Shigella bacterium may comprise hybrid O-antigen, a mixture of at least two different O-antigens, or a combination of hybrid O-antigen and a mixture of O-antigens.

If the outer membrane vesicle or the recombinant Shigella bacterium comprises at least one hybrid O-antigen, then the outer membrane vesicle or the recombinant Shigella bacterium comprises at least one O-antigen species in which epitopes from at least two different serotypes are present on the same molecule. For example, as discussed above S. flexneri serotype 1a is characterised by O-antigen comprising an epitope comprising α(1-4) glucosylation of GlcNAc and S. flexneri serotype 2a is characterised by O-antigen comprising an epitope comprising α(1-4) glucosylation of RhaI. It is possible for a single molecule of O-antigen to comprise both α(1-4) glucosylation of GlcNAc and α(1-4) glucosylation of RhaI, and such O-antigen would be considered to be a hybrid O-antigen comprising epitopes from S. flexneri serotypes 1a and 2a. Some O-antigen that occur in nature may be considered to be hybrid O-antigen. For example, O-antigen from serotype 1d may be considered to comprise epitopes from serotypes 1a and x, optionally, the hybrid O-antigen is not an O-antigen that occurs in nature, for example optionally the hybrid O-antigen is not an O-antigen of serotype 1d, 1b, 2b, 4b, 5b or 3a.

If the outer membrane vesicle or the Shigella bacterium comprises a mixture of at least two different O-antigens it comprises multiple different O-antigen species, comprising different O-antigen comprising epitopes from different Shigella bacteria. For example, in analogy to the example provided above, Shigella bacteria or outer membrane vesicles comprising some O-antigen molecules comprising α(1-4) glucosylation of GlcNAc and different O-antigens molecules comprising α(1-4) glucosylation of RhaI would be considered to be Shigella bacteria or outer membrane vesicles comprising a mixture of O-antigens from serotype 1a and serotype 2a.

In some embodiments, the outer membrane vesicle or recombinant Shigella bacterium comprises epitopes from at least three different Shigella serotypes, for example at least four different S. flexneri serotypes.

The O-antigen epitopes described above for various S. flexneri strains are all characteristic glucosylation or O-acetylation patterns (for example α(1-4) glucosylation of GlcNAc is glucosylation of O-antigen at the α(1-4) and may be considered to be a glucosylation pattern). Thus, the epitopes from at least two different serotypes of Shigella may comprise glucosylation or O-acetylation patterns characteristic of at least two different serotypes of Shigella flexneri.

### Hybrid O-Antigen

As described above, the Shigella bacteria or outer membrane vesicles of the invention may comprise hybrid O-antigen, i.e. the outer membrane vesicle of the recombinant Shigella bacterium may comprise O-antigen in which epitopes from at least two different Shigella bacteria are present on the same molecule. Optionally, the O-antigen comprising epitopes from at least two different serotypes of Shigella or S. flexneri comprises at least one hybrid O-antigen. Hybrid O-antigen may be produced by providing Shigella bacteria with genes encoding enzymes that modify Shigella O-antigen to introduce the glucosylation and/or O-acetylation patterns from multiple different strains of Shigella. However, as set out in more detail in the Examples, some enzymes that modify Shigella O-antigen are not compatible with one another. Without being bound by theory, it seems that some enzymes may struggle to modify the same molecule of O-antigen, either because they sterically hinder one another or because the modification introduced by one enzyme modifies the site at which the second enzyme normally introduces modifications. In such cases, providing Shigella bacteria with genes for these two enzymes will result in a mixture of O-antigen (rather than hybrid O-antigen) as described in more detail below. Whether or not different combinations of the enzymes gtrI, gtrII, gtrIV, gtrV, gtrX and oacA are compatible with one another is set out in Table 8. Enzyme combinations that produce greater than 30% hybrid O-antigen are considered compatible (either fully or partially compatible). Enzyme combinations that produce greater than 80% hybrid O-antigen are considered to be fully compatible (e.g. gtrI and gtrX, gtrI and gtrV, gtrI and oacA, gtrII and gtrX, gtrX and gtrV, gtrX and oacA, and gtrIV and oacA). Enzymes that produce hybrid O-antigen but less than 80% hybrid O-antigen (the rest is a mixture of different O-antigen) are considered to be partially compatible (e.g. gtrII and gtrIV, gtrII and gtrV, gtrX and gtrIV, and gtrIV and gtrV).

Optionally, the hybrid O-antigen comprises epitopes from at least two or at least three different serotypes of Shigella. Optionally, the hybrid O-antigen comprises epitopes from two, three or four different S. flexneri serotypes.

Optionally, the outer membrane vesicle or recombinant Shigella bacterium comprises hybrid O-antigen that comprises epitopes from S. flexneri serotypes (i) 1a and 5a, (ii) 1a and X (i.e. serotype 1d), (iii) 1a and 3b (i.e. serotype 1b), (iv) 1a, 5a and X, (v) 1a, X and 3b, (vi) 2a and 4a, (vii) 2a and 5a, (viii) 2a and X (i.e. serotype 2b), (ix) 2a, 4a and 5a, (x) 2a, 4a and X, (xi) 2a, 5a and X, (xii) 2a, 4a, 5a and X, (xiii) 4a and 5a, (xiv) 4a and X, (xv) 4a and 3b (i.e. serotype 4b), (xvi) 4a, 5a and X, (xvii) 4a, 3b and X, (xviii) 5a and X (i.e. serotype 4b), or (xix) X and 3b (i.e. serotype 3a).

As discussed above, α(1-4) glucosylation of GlcNAc is the O-antigen epitope from serotype 1a, α(1-4) glucosylation of RhaI is the O-antigen epitope from serotype 2a, α(1-6) glucosylation of GlcNAc is the O-antigen epitope from serotype 4a, α(1-3) glucosylation of RhaII is the O-antigen epitope from serotype 5a, α(1-4) glucosylation of RhaIII is the O-antigen epitope from serotype x and 2-O-Acetylation of RhaI is the O-antigen epitope from serotype 3b. Thus, if the Shigella bacterium or the outer membrane vesicle comprises O-antigen, comprising an O-antigen species in which α(1-4) glucosylation of RhaI and α(1-3) glucosylation of RhaII is present on the same O-antigen molecule, then the Shigella bacterium or the outer membrane vesicle comprises a hybrid O-antigen that comprises epitopes from serotypes 1a and 5a.

Optionally, the hybrid O-antigen comprises epitopes from S. flexneri serotypes (i) 1a and 5a, (ii) 1a, 5a and X, (iii) 1a, X and 3b, (iv) 2a and 4a, (v) 2a and 5a, (vi) 2a, 4a and 5a, (vii) 2a, 4a and X, (viii) 2a, 5a and X, (ix) 2a, 4a, 5a and X, (x) 4a and 5a, (xi) 4a and X, (xii) 4a, 5a and X, or (xiii) 4a, 3b and X.

Optionally, the hybrid O-antigen comprises epitopes from S. flexneri serotypes (i) 1a and 5a, (ii) 1a and X, (iii) 1a and 3b, (iv) 2a and 4a, (v) 2a and 5a, (vi) 2a and X, (vii) 4a and 5a, (viii), 4a and X, (ix) 5a and X, (x) 4a and 3b, or (xi) 3b and X. Optionally, the hybrid O-antigen comprises epitopes from S. flexneri serotypes 1a and 2b.

Optionally, the hybrid O-antigen comprises epitopes from S. flexneri serotypes 1a and 3b or 4a and 3b.

Optionally, the hybrid O-antigen comprises epitopes from S. flexneri serotypes (i) 1a and 5a, (ii) 1a and X, (iii) 1a and 3b, (iv) 1a, 5a and X, (v) 1a, X and 3b, (vi) 2a and X, (vii) 4a and 3b, (viii) X and 5a, or (ix) X and 3b.

### A Mixture of O-Antigen

As described above, the outer membrane vesicles or Shigella bacteria may comprise a mixture of O-antigen, i.e. they may comprise multiple different O-antigen species, comprising different O-antigen comprising epitopes from different Shigella bacteria. A mixture of O-antigen may arise when Shigella bacteria are cultured with genes that encode two enzymes that modified Shigella O-antigen that are not compatible with one another (as explained above in the section entitled “Hybrid O-antigen”. For example, gtrI and gtrII, gtrI and gtrIV, gtrII and oacA, gtrV and oacA are not compatible with one another.

The mixture may comprise at least two different O-antigens from at least two different Shigella flexneri serotypes. The mixture may comprise epitopes from S. flexneri serotypes (i) 1a and 2a, (ii) 1a and 4a, (iii) 2a and 4a, (iv) 2a and 5a, (v) 2a and 3b, (vi) X and 4a, (vii) 4a and 5a, and/or (viii) 5a and 3b.

Optionally, the mixture may comprise epitopes from S. flexneri serotypes (i) 1a and 2a, (ii) 1a and 4a, (iii) 2a and 3b, and/or (iv) 5a and 3b.

Optionally, the mixture may comprise epitopes from S. flexneri serotypes (i) 1a, 2a and X, (ii) 1a, 2a and 4a, (iii) 1a, 2a and 5a, (iv) 1a, 2a and 3b, (v) 1a, 2a, X and 4a, (vi) 1a, 2a, X and 5a, (vii) 1a, 2a, X and 3b, (viii) 1a, 2a, 4a and 5a, (ix) 1a, 2a, 4a and 3b, (x) 1a, 2a, 5a and 3b, (xi) 1a, 2a, X, 4a and 5a, (xii) 1a, 2a, X, 4a and 3b, (xiii) 1a, 2a, X, 5a and 3b, (xiv) 1a, 2a, 4a, 5a and 3b, (xv) 1a, 2a, X, 4a, 5a and 3b, (xvi) 1a, 4a and X, (xvii) 1a, 4a and 5a, (xviii) 1a, 4a and 3b, (xix) 1a, 4a, X and 5a, (xx) 1a, 4a, X and 3b, (xxi) 1a, 4a, 5a and 3b, (xxii) 1a, 4a, X, 5a and 3b, (xxiii) 2a, 3b and X, (xxiv) 2a, 3b and 4a, (xxv) 2a, 3b and 5a, (xxvi) 2a, 3b, X and 4a, (xxvii) 2a, 3b, X and 5a, (xxviii) 2a, 3b, 4a and 5a, (xxix) 2a, 3b, X, 4a and 5a, (xxx) 5a, 3b and 1a, (xxxi) 5a, 3b and X, (xxxii) 5a, 3b and 4a, (xxxiii) 5a, 3b, 1a and X, and/or (xxxiv) 5a, 3b, X and 4a.

### Methods for Preparing Shigella Outer Membrane Vesicles or a Recombinant Shigella Bacterium Comprising at Lest One Heterologous O-Antigen

The present invention provides methods for preparing Shigella outer membrane vesicles. In some aspects, the method for preparing Shigella outer membrane vesicles comprises:
(i) providing at least one gene encoding an enzyme that modifies a Shigella O-antigen on at least one plasmid;
(ii) culturing a production Shigella bacterium in the presence of the at least one plasmid under conditions suitable for the enzyme to be expressed by the recombinant Shigella bacterium and for Shigella outer membrane vesicles to be produced; and
(iii) harvesting the outer membrane vesicles.

The present invention also provides, a method for preparing a recombinant Shigella bacterium comprising at least one heterologous O-antigen comprising:
(i) providing at least one gene encoding an enzyme that modifies a Shigella O-antigen on at least one plasmid;
(ii) culturing a production Shigella bacterium in the presence of the at least one plasmid under conditions suitable for the enzyme to be expressed by the production Shigella bacterium to produce the recombinant Shigella bacterium comprising at least one heterologous O-antigen; and
(iii) harvesting the recombinant Shigella bacterium comprising at least one heterologous O-antigen.

As discussed in Table 1 above, there are various genes that are known to modify a Shigella O-antigen, in particular genes are known that modify the glucosylation and O-acetylation patterns of Shigella O-antigen. For example the gtrI gene encodes an enzyme that catalyses α(1-4) glucosylation of GlcNAc, gtrII encodes an enzyme that catalyses α(1-4) glucosylation of RhaI, gtrIV encodes an enzyme that catalyses α(1-6) glucosylation of GlcNAc, gtrV encodes an enzyme that catalyses α(1-3) glucosylation of RhaII, gtrX encodes an enzyme that catalyses α(1-4) glucosylation of RhaIII and oacA encodes an enzyme that catalyses 2-O-Acetylation of RhaI. These glucosylation and O-acetylation patterns serve as epitopes characteristic of different serotypes of Shigella, for example S. flexneri. As discussed in the summary of the invention, in the present methods, plasmids comprising genes that modify a Shigella O-antigen may be used to prepare Shigella bacteria which comprise a modified Shigella O-antigen in their outer membrane, and which release outer membrane vesicles comprising the modified Shigella O-antigen. Such methods may be used to generate a recombinant Shigella bacterium, and outer membrane vesicles comprising heterologous O-antigen such as hybrid O-antigen or a mixture of O-antigen. Such methods are advantageous, as they allow the user to take a well-characterized and safe strain of Shigella, and use that strain to produce vaccines against a range of different Shigella serotypes more quickly and easily that using prior art methods.

Optionally, the methods comprise a step prior to step (i) of selecting an at least one gene. Optionally, the step of selecting an at least one gene comprises a step of selecting the O-antigen epitopes that are desired and selecting genes that modify O-antigen to comprise the selected epitopes. Suitable enzymes are set out in Table 1 and discussed further below. For example, if the user wishes to select a serotype 2a O-antigen epitope, then the user will select a gtrII gene as the at least one gene. Optionally, the step of selecting the O-antigen epitopes that are desired comprises determining the serotype of circulating Shigella strains, for example circulating S. flexneri strains and selecting the O-antigen epitopes of those circulating strains.

Optionally, the method is a method for preparing Shigella outer membrane vesicles comprising O-antigen comprising epitopes from at least two, or at least three different serotypes of S. flexneri. Optionally, the method is a method for preparing a recombinant Shigella bacterium comprising O-antigen comprising epitopes from at least two, or at least three different serotypes of S. flexneri. Optionally, the method is a method for preparing Shigella outer membrane vesicles or a recombinant Shigella bacterium of the invention.

Plasmid Comprising at Least One Gene Encoding an Enzyme that Modifies a Shigella O-Antigen

As set out above, the present invention relates to a plasmid comprising at least one gene encoding an enzyme that modifies a Shigella O-antigen. The present invention also relates to methods comprising providing at least one gene encoding an enzyme that modifies a Shigella O-antigen on at least one plasmid.

The term “plasmid” is well known in the art, and may refer to any circular strand of DNA. The skilled person is aware of methods of introducing a gene of interest, for example a gene encoding an enzyme that modifies a Shigella O-antigen, into a plasmid sequence. For example, various plasmid backbones containing a variety of restriction enzyme sites are readily available. The pCOLA-Duet and/or pACUC-Duet plasmids may be used.

Optionally, the at least one gene is under the control of a promoter i.e. the at least one plasmid comprises at least one promoter operably linked to each or all of the at least one genes. The promoter may be any suitable promoter, for example a Shigella promoter (i.e. a promoter found in the native Shigella genome), such as an S. flexneri promoter (i.e. a promoter found in the native S. flexneri genome). Optionally, the promoter is a constitutive promoter. Optionally, the promoter is the native promoter. For example, as set out in more detail below, the at least one gene may comprise a gtrI gene, and in such embodiments the gtrI gene may be operably linked to a gtrI promoter. If one at least one plasmid comprises two or more genes encoding an enzyme that modifies a Shigella O-antigen, all the genes may be under the control of a single promoter which may be the native promoter of one of the genes.

The native promoters of the gtrI, gtrII, gtrIV, gtrV, gtrX, oacA, oacB and oacD genes are set out in SEQ ID NOs: 9 to 18.

Enzyme that Modifies a Shigella O-Antigen on at Least One Plasmid

As set out above, the present invention relates to methods comprising providing at least one gene encoding an enzyme that modifies a Shigella O-antigen on at least one plasmid.

The at least one gene may comprise at least 2, at least 3, at least 4, between 1 and 6, or between 1 and 5 gene(s). If more than one at least one gene is used, the genes may be provided on the same plasmid or may be provided on multiple different plasmids. In some embodiments, no more than 2 of the at least one genes are present on the same plasmid. The at least one plasmid may comprise at least 1, at least 2, or around 2 plasmids.

Optionally, the at least one gene comprises at least 1, at least 2, at least 3, between 1 and 5 genes, or between 1 and 4 genes encoding a glucosyltransferase. Optionally, the at least one gene encoding a glucosyltransferase comprises 1, 2, 3, 4 or 5 genes selected from the group consisting of gtrI, gtrII, gtrIV, gtrV and gtrX.

Optionally, if the at least one gene comprises the gtrI gene, then the gtrI gene is operably linked to the gtrI gene promoter (e.g. the promoter of SEQ ID NO: 9). Optionally, if the at least one gene comprises the gtrII gene, then the gtrII gene is operably linked to the gtrII gene promoter (e.g. the promoter of SEQ ID NO: 10). Optionally, if the at least one gene comprises the gtrIV gene, then the IV gene is operably linked to the gtrIV gene promoter (e.g. the promoter of SEQ ID NO: 11). Optionally, if the at least one gene comprises the gtrV gene, then the gtrV gene is operably linked to the gtrV gene promoter (e.g. the promoter of SEQ ID NO: 12). Optionally, if the at least one gene comprises the gtrX gene, then the gtrX gene is operably linked to the gtrX gene promoter (e.g. the promoter of SEQ ID NO: 13).

Optionally, the at least one gene comprises at least 1, at least 2, or between 1 and 3 gene(s) encoding an O-antigen O-acetylase. Optionally, the at least one gene encoding an O-antigen O-acetylase comprises 1, 2 or 3 genes selected from the group consisting of oacA, oacB, and oacD.

Optionally, if the at least one gene comprises the oacA gene, then the oacA gene is operably linked to the oacA gene promoter (e.g. the promoter of SEQ ID NO: 14).

Optionally, the at least one gene comprises (i) gtrI and gtrII, (ii) gtrI and gtrV, (iii) gtrI and gtrX, (iv) gtrI and oacA, (v) gtrII and gtrV, (vi) gtrII and gtrX, (vii) gtrII and oacA, (viii) gtrIV and gtrI, (ix) gtrIV and gtrII, (x) gtrIV and gtrV, (xi) gtrIV and gtrX, (xii) gtrIV and oacA, (xiii) gtrV and gtrX, (xiv) gtrV and oacA, or (xv) gtrX and oacA.

Optionally, the at least one gene comprises (i) gtrI and gtrII, (ii) gtrI and gtrV, (iii) gtrII and gtrV, (iv) gtrII and oacA, (v) gtrIV and gtrI, (vi) gtrIV and gtrII, (vii) gtrIV and gtrV, (viii) gtrIV and gtrX, or (ix) gtrV and oacA.

Optionally, the at least one gene comprises (i) gtrI and gtrII, (ii) gtrI and gtrV, (iii) gtrI and gtrX, (iv) gtrI and oacA, (v) gtrII and gtrV, (vi) gtrII and gtrX, (vii) gtrII and oacA, (viii) gtrIV and gtrI, (ix) gtrIV and gtrII, (x) gtrIV and gtrV, (xi) gtrIV and gtrX, (xii) gtrIV and oacA, (xiii) gtrV and gtrX, (xiv) gtrV and oacA, or (xv) gtrX and oacA; and the recombinant Shigella bacterium is a bacterium whose genome has been modified to delete one or more of the following genes that are present in the native genome: gtrI, gtrII, gtrIV, gtrV, gtrX, OacA, OacB and OacD.

Optionally, the at least one gene comprises (i) gtrI and gtrII, (ii) gtrI and gtrV, (iii) gtrI and gtrX, (iv) gtrI and oacA, (v) gtrII and gtrV, (vi) gtrII and gtrX, (vii) gtrII and oacA, (viii) gtrIV and gtrI, (ix) gtrIV and gtrII, (x) gtrIV and gtrV, (xi) gtrIV and gtrX, (xii) gtrIV and oacA, (xiii) gtrV and gtrX, (xiv) gtrV and oacA, or (xv) gtrX and oacA; and the recombinant Shigella bacterium is a Shigella flexneri serotype 2a bacterium that has been modified by deleting the OacD, OacB and gtrII genes.

Optionally, the at least one gene comprises (i) gtrI and gtrII, (ii) gtrI and gtrV, (iii) gtrI and gtrX, (iv) gtrI and oacA, (v) gtrII and gtrV, (vi) gtrII and gtrX, (vii) gtrII and oacA, (viii) gtrIV and gtrI, (ix) gtrIV and gtrII, (x) gtrIV and gtrV, (xi) gtrIV and gtrX, (xii) gtrIV and oacA, (xiii) gtrV and gtrX, (xiv) gtrV and oacA, or (xv) gtrX and oacA; and the recombinant Shigella bacterium is a Shigella flexneri serotype 2a bacterium that has been modified by deleting the OacD, OacB and gtrII genes, and the at least one plasmid comprises pCOLA-Duet or pACYC-Duet backbone.

Optionally, the at least one gene comprises (i) gtrI and gtrII, (ii) gtrI and gtrV, (iii) gtrII and gtrV, (iv) gtrII and oacA, (v) gtrIV and gtrI, (vi) gtrIV and gtrII, (vii) gtrIV and gtrV, (viii) gtrIV and gtrX, or (ix) gtrV and oacA; and the recombinant Shigella bacterium is a bacterium whose genome has been modified to delete one or more of the following genes that are present in the native genome: gtrI, gtrII, gtrIV, gtrV, gtrX, OacA, OacB and OacD.

Optionally, the at least one gene comprises (i) gtrI and gtrII, (ii) gtrI and gtrV, (iii) gtrII and gtrV, (iv) gtrII and oacA, (v) gtrIV and gtrI, (vi) gtrIV and gtrII, (vii) gtrIV and gtrV, (viii) gtrIV and gtrX, or (ix) gtrV and oacA; and the recombinant Shigella bacterium is a Shigella flexneri serotype 2a bacterium that has been modified by deleting the OacD, OacB and gtrII genes.

Optionally, the at least one gene comprises (i) gtrI and gtrII, (ii) gtrI and gtrV, (iii) gtrII and gtrV, (iv) gtrII and oacA, (v) gtrIV and gtrI, (vi) gtrIV and gtrII, (vii) gtrIV and gtrV, (viii) gtrIV and gtrX, or (ix) gtrV and oacA; and the recombinant Shigella bacterium is a Shigella flexneri serotype 2a bacterium that has been modified by deleting the OacD, OacB and gtrII genes, and the at least one plasmid comprises pCOLA-Duet or pACYC-Duet backbone.

Optionally, the at least one gene comprises (i) gtrI, (ii) gtrII, (iii) gtrIV, (iv) gtrV, (vi) gtrI and gtrII, (vii) gtrIV and gtrV, or (viii) gtrI, gtrII, gtrIV and gtrV. Optionally, the at least one gene comprises (i) gtrI, (ii) gtrII, (iii) gtrIV, (iv) gtrV, (vi) gtrI and gtrII, (vii) gtrIV and gtrV, or (viii) gtrI, gtrII, gtrIV and gtrV and the recombinant Shigella bacterium is a recombinant S. flexneri serotype 3a Shigella bacterium. Optionally, the at least one gene comprises (i) gtrI, (ii) gtrII, (iii) gtrIV, (iv) gtrV, (vi) gtrI and gtrII, (vii) gtrIV and gtrV, or (viii) gtrI, gtrII, gtrIV and gtrV, the recombinant Shigella bacterium is a recombinant S. flexneri serotype 3a Shigella bacterium, and the at least one plasmid comprises pCOLA-Duet or pACYC-Duet backbone.

### Culturing a Production Shigella Bacterium in the Presence of at Least One Plasmid Under Conditions

Methods of the invention may comprise a step of culturing a production Shigella bacterium in the presence of the at least one plasmid under conditions suitable for the enzyme to be expressed by the production Shigella bacterium.

As discussed above, the at least one plasmid comprises genes encoding enzymes that modify Shigella O-antigen. Suitable such genes, like gtrI, gtrII, gtrIV, gtrV, gtrX, and OacA can modify the Shigella O-antigen so that it comprises epitopes of specific strains of S. flexneri, as discussed above and set out in Table 1. If a production Shigella bacterium is cultured in the presence of the at least one plasmid, the at least one plasmid may complement the Shigella bacterium and the transcription and translation machinery of the Shigella bacterium may cause expression of the at least one enzyme. Thus, if a production Shigella bacterium is cultured in the presence of at least one plasmid under conditions suitable for the enzyme to be expressed by the production Shigella bacterium, the O-antigen of the production Shigella bacterium may be modified by the enzyme such that it comprises heterologous epitopes from one or more Shigella strains.

The skilled person is aware of conditions that are suitable for culturing Shigella bacteria, and conditions that are suitable for an enzyme to be expressed from at least one plasmid by Shigella bacteria. For example, the skilled person is aware of how to transform cells with plasmids such as the pACYC-Duet and pCOLA-Duet plasmids. Similarly, the skilled person is aware of suitable conditions for culturing a Shigella bacterium. Suitably the production Shigella bacterium can be cultured using the same or similar culture conditions to those set out in Example 2. Suitably, the production Shigella bacteria are cultured as described under the heading “Bacterial strains and growth condition”. For example, the production Shigella bacteria may be grown in Luria-Bertani medium, or in HTMC medium (15 g/L Glycerol, 30 g/L Yeast extract, 0.5 g/L MgSO₄, 5 g/L KH₂PO₄, 20 g/L K₂HPO₄) for outer membrane vesicle production.

Once the production Shigella bacterium has been cultured in the presence of the at least one plasmid under conditions suitable for the enzyme to be expressed by the production Shigella bacterium, the production Shigella bacterium will begin to comprise a heterologous O-antigen in its outer membrane, specifically a heterologous O-antigen comprising epitopes from specific Shigella serotypes depending on the nature of the enzymes encoded by the at least one genes, i.e. the production Shigella bacterium becomes a recombinant Shigella bacterium comprising at least one heterologous O-antigen. The recombinant Shigella bacterium comprising at least one heterologous O-antigen may be harvested and, for example, used in a vaccine. Alternatively, the recombinant Shigella bacterium may be cultured in conditions suitable for Shigella outer membrane vesicles to be produced. The conditions suitable for the enzyme to be expressed by the recombinant Shigella bacterium may be different compared to the conditions suitable for Shigella outer membrane vesicles to be produced, for example, the medium may be changed from a medium that promotes Shigella growth to a medium that promotes outer membrane vesicle production. However, in methods for preparing Shigella outer membrane vesicles, both conditions suitable for the enzyme to be expressed by the recombinant Shigella bacterium and conditions suitable for Shigella outer membrane vesicles to be produced should be used.

### Production Shigella Bacterium

The methods of the invention comprise culturing a production Shigella bacterium. Any suitable Shigella bacterium may be used. As described above, Shigella are Gram-negative non-motible facultative anaerobic bacilli that fall into four serogroups S. dysenteriae, S. flexneri, S. boydii and S. sonnei. S. dysenteriae, S. flexneri, S. boydii and S. sonnei express O-antigen in the outer membrane.

In a preferred embodiment, the production Shigella bacterium is an S. flexneri bacterium. There are several different serotypes of S. flexneri distinguished by the nature of the O-antigen expressed by the bacterium. All serotypes of S. flexneri, except serotype 6, share a conserved polysaccharide backbone (corresponding to serotype Y) consisting of the following OAg repeating unit (RU): →2)-α-L-RhapIII-(1→2)-α-L-RhapII-(1→3)-α-L-RhapI-(1→3)-β-D-GlcpNAc-(1→.

The diversity of S. flexneri serotypes is due to the modification of this common OAg backbone with glucosyl and/or O-Acetyl groups, as a result of bacteriophages infection and acquisition of OAg-modifying enzymes.

Optionally, the production Shigella bacterium comprises a genome encoding the enzymes required for expressing an S. flexneri O-antigen selected from the group consisting of serotype 1a, serotype 1d, serotype 2a, serotype 3a, serotype 3b, serotype 4a, serotype 4b, serotype 5a, serotype X, and serotype Y. As discussed above, any suitable Shigella bacterium may be used as the production Shigella bacterium, and that includes Shigella that naturally express O-antigen comprising epitopes of a particular Shigella serotype.

In such embodiments, optionally the production Shigella bacterium is an S. flexneri bacterium of serotype 1a, serotype 1d, serotype 2a, serotype 3a, serotype 3b, serotype 4a, serotype 4b, serotype 5a, serotype X, and serotype Y. Whether or not a strain comprises a genome encoding the enzymes required for expressing an S. flexneri O-antigen selected from the group consisting of serotype 1a, serotype 1d, serotype 2a, serotype 3a, serotype 3b, serotype 4a, serotype 4b, serotype 5a, and serotype X or is an S. flexneri bacterium of serotype 1a, serotype 1d, serotype 2a, serotype 3a, serotype 3b, serotype 4a, serotype 4b, serotype 5a, serotype X, and serotype Y may be determined using methods known in the art. For example, the user may grow the production Shigella bacterium in conditions suitable for O-antigen production (which are synonymous with the “conditions suitable for the enzyme to be expressed” which are discussed further below), and determine the nature of the O-antigen that is produced. The skilled person may determine the identity of the OAg by using a combination of HPAEC-PAD (to determine sugar content) and GLC-MS to determine the linkage positions for the consistent sugars. Alternatively, the skilled person could use ¹H-NMR to determine the O-antigen identity. If the production Shigella bacterium produces a serotype 2a O-antigen under conditions suitable for O-antigen production, then the production Shigella bacterium comprises a genome encoding the enzyme required for expressing an S. flexneri O-antigen from serotype 2a, or the production Shigella bacterium is an S. flexneri bacterium of serotype 2a. Optionally, the production Shigella bacterium is an S. flexneri bacterium of serotype 2a or 3a.

Optionally, the production Shigella bacterium is a bacterium whose genome has been modified to delete one or more of the following genes that are present in the native genome: gtrI, gtrII, gtrIV, gtrV, gtrX, oacA, oacB and oacD. Optionally, the production Shigella bacterium is a bacterium whose genome has been modified to delete any of the following genes that are present in the native genome: gtrI, gtrII, gtrIV, gtrV, gtrX, oacA, oacB and oacD. As noted above, any suitable Shigella strain may be used as the production strain. The user may choose to select a Shigella strain that is well characterised such as a serotype 2a strain, which is discussed further below. If the production Shigella bacterium comprises a genome encoding one or more of GtrI, GtrII, GtrIV, GtrV, GtrX, OacA, OacB and OacD, then the at least one plasmid can be used to produce recombinant Shigella bacteria or outer membrane vesicles comprising epitopes from the production strain itself and additional epitopes that are added to the O-antigen by the enzymes encoded by the at least one gene. For example, if the production Shigella bacterium comprises an S. flexneri serotype 2a bacterium, then a plasmid comprising a gene encoding gtrI could be used to prepare recombinant Shigella bacteria or outer membrane vesicles comprising O-antigen comprising epitopes from serotype 2a and 1a. However, there may be situations where the user does not want the O-antigen to comprise epitopes from serotype 2a. For example, as set out above, some of the enzymes that modify O-antigen are not compatible with one another and if the user wanted to produce outer membrane vesicles comprising homogenous hybrid O-antigen he would not be able to do that using a serotype 2a strain complemented by a plasmid comprising gtrI (for example). However, that can be remedied by modifying the genome of the production Shigella strain to remove at least one or all genes that encode enzymes that modify O-antigen to produce a “scaffold” or serogroup Y Shigella bacterium.

Suitably, the production Shigella bacterium is an S. flexneri serotype 2a bacterium that has been modified by deleting the OacD, OacB and gtrII genes. Vaccines against S. flexneri serotype 2, comprising antigens from S. flexneri serotype 2a are being developed, and so there are a number of well characterised S. flexneri serotype 2a strains that could be used as the production Shigella bacterium. S. flexneri serotype 2a comprises O-antigen that is modified by the OacD, OacB and gtrII genes, so by modifying an S. flexneri serotype 2a bacterium to remove these genes, the user may prepare a scaffold (serogroup Y) Shigella bacterium based on a well-characterised parent Shigella strain.

Optionally, the method comprises steps used to prepare suitable production Shigella bacteria. For example, the method may comprise a step of preparing the production Shigella bacterium, prior to step (i), by modifying a Shigella bacterium to express a heterologous O-antigen. Optionally, the step of preparing the production Shigella bacterium comprises or consists of deleting one or more of the following genes that are present in the native genome: gtrI, gtrII, gtrIV, gtrV, gtrX, oacA, oacB and oacD.

Optionally, the step of preparing the production Shigella bacterium comprises or consists of modifying a Shigella flexneri serotype 2a bacteria by deleting the oacD, oacB and gtrII genes. Deleting genes may be achieved using any technique known in the art. For example, a suitable technique is described in Example 2.

### Hyber-Blebbing

Alternatively or additionally, the recombinant Shigella bacterium or the production Shigella bacterium may comprise at least one mutation that causes it to release greater quantities of outer membrane vesicles, sometimes referred to as a “hyber-blebbing” mutation. Optionally, the at least one mutation or the hyber-blebbing mutation causes the recombinant Shigella bacterium or the production Shigella bacterium to release greater quantities of outer membrane vesicles compared to an equivalent (otherwise identical) Shigella bacterium not comprising the at least one mutation or at least one hyper-blebbing mutations. The rate at which the recombinant Shigella bacterium and the equivalent bacterium release outer membrane vesicles may be evaluated by FM4-64 fluorescence, normalising each fluorescence value to the optical density of the corresponding clone. Alternatively, optionally the rate may be determined by measuring release of outer membrane vesicles using microBCA to measure outer membrane vesicle (OMV) total protein content, and normalising the total protein content by dividing it by the volume of the supernatant. In such methods, the yield or rate is expressed in milligrams of OMV per liter of supernatant.

Optionally, the recombinant Shigella bacterium or the production Shigella bacterium comprises a mutation that inactivates tolR, such as a deletion of all or a portion of the tolR gene. “Inactivation” in the context of a gene refers to mutating or deleting the gene such that the protein to be transcribed can no longer carry out the function of the corresponding wild-type protein, or carries out the function to a lesser extent.

The skilled person can determine whether a mutation inactivates tolR, by comparing a recombinant Shigella bacterium that comprises the tolR mutation, with an equivalent Shigella bacterium that does not comprise the tolR mutation and determining whether the recombinant Shigella bacterium that comprises the mutation releases greater quantities of outer membrane vesicles compared to the equivalent Shigella bacterium using the FM4-64 fluorescence assay described in the preceding paragraph. If the recombinant Shigella bacterium that comprises the mutation does release greater quantities of outer membrane vesicles compared to the equivalent Shigella bacterium, then the tolR mutation is one that inactivates tolR.

### Harvesting and Purifying the Outer Membrane Vesicles

The methods of the invention may comprise a step of harvesting outer membrane vesicles. Any suitable technique for harvesting outer membrane vesicles may be used. For example, outer membrane vesicles may be harvested by detergent extraction. Alternatively, as described briefly above, the production Shigella bacterium and/or the recombinant Shigella bacterium of the invention may be modified to improve the rate at which they spontaneously release outer membrane vesicles. In methods in which such “hyper blebbing” Shigella bacteria are used (as the production Shigella bacterium), the outer membrane vesicles produced may be harvested by collection of cell culture medium in which the production Shigella bacterium is cultured.

The methods of the invention may comprise a further step of purifying the outer membrane vesicles. Outer membrane vesicles can be purified from the culture medium. The purification ideally involves separating the outer membrane vesicles from living and/or intact Shigella bacteria, for example, by size-based filtration using a filter, such as a 0.2 μm filter, which allows the outer membrane vesicles to pass through but which does not allow intact bacteria to pass through, or by using low speed centrifugation to pellet cells while leaving outer membrane vesicles in suspension. Suitable purification methods are known in the art. A preferred two-step filtration purification process is described in WO 2011/036562 herein incorporated by reference. Particularly the two-step filtration process is used to separate outer membrane vesicles from cell culture biomass without using centrifugation.

### Harvesting and Purifying the Recombinant Shigella Bacterium

The methods of the invention may comprise a step of harvesting the recombinant Shigella bacterium. Any suitable technique for harvesting bacteria may be used.

The methods of the invention may comprise a further step of purifying the recombinant Shigella bacterium comprising at least one heterologous O-antigen.

### Detoxification

The outer membrane vesicle, recombinant Shigella bacterium or production Shigella bacterium may comprise modified lipid. Optionally, the modified lipid A is less toxic compared to a corresponding lipid A.

“Toxicity” or “toxic” in this context refers to the extent to which the innate immune system is activated by lipid A, particularly through the Toll-like receptor 4 pathway. Highly toxic lipid A can lead to uncontrolled inflammation, apoptosis, and in extreme cases septic shock, among other effects. Therefore, a “less toxic” lipid A refers to a lipid A that activates the Toll-like receptor 4 pathway to a lesser extent relative to a corresponding wild-type lipid A, for example by having a structure that is less likely to be recognised by Toll-like receptor 4. Optionally, a modified lipid A is less toxic if it is less reactogenic than a corresponding wild-type lipid A. For example, one can determine whether a modified lipid A is less toxic by administering it to an animal such as a rabbit or mouse, and determine whether it activated more monocytes compared to a corresponding wild-type lipid A.

“Corresponding wild-type lipid A” refers to lipid A that can be found in the corresponding wild-type bacterium and strain. For example, lipid A that is modified relative to a “corresponding wild-type lipid A” in the context of Shigella flexneri serotype 1b GMMA is interpreted to mean a lipid A that is modified (e.g. such that it is less toxic) relative to lipid A found in wild-type Shigella flexneri serotype 1b.

In an embodiment, the recombinant Shigella bacterium and/or the production Shigella bacterium comprises one or more mutations resulting in inactivation of htrB, msbB1 and/or msbB2. The outer membrane vesicles of the invention may be produced from a production Shigella bacterium comprising one or more mutations resulting in inactivation of htrB, msbB1 and/or msbB2. Inactivation of htrB, msbB1 and msbB2 results in Shigella that produced outer membrane vesicles comprising lipid A that is less toxic than wild-type lipid A.

By way of non-limiting example, suitable recombinant Shigella bacteria and/or the production Shigella bacteria may be selected from the group consisting of ΔhtrB, ΔmsbB1 and ΔmsbB2 bacteria (ΔhtrB refers to a Shigella strain which has the htrB gene deleted). For simplicity, double deletions of both msbB1 and msbB2 may also be referred to as ΔDmsbB. Inactivation of htrB or msbB1 and msbB2 reduce acylation in lipid A. Optionally, inactivation of msbB1 and/or msbB2 is used.

As noted above, “Inactivation” in the context of a gene refers to mutating or deleting the gene such that the protein to be transcribed can no longer carry out the function of the corresponding wild-type protein, or carries out the function to a lesser extent. For example, “inactivation of htrB” refers to deleting the gene, or mutating the gene, in a Shigella bacterium such that acylation of lipid A in the Shigella bacterium is reduced. Optionally, whether or not a Shigella bacterium include one or mutations resulting in “inactivation” of htrB, or an msbB protein may be determined by isolating outer membrane vesicles (e.g. GMMA) from the Shigella bacterium and analysing the lipid A in the outer membrane vesicles using MALDI-TOF analysis. One may compare the spectrum generated by MALDI-TOF analysis with a spectrum produced by analysing lipid A from Shigella bacteria that comprise wild type htrB and msbB genes, and if the amount of hexa-acylation is reduced, then the Shigella bacterium includes one or mutations resulting in inactivation of htrB and/or msbB.

The modified lipid A may have a reduced level of hexa-acylated lipid A. The recombinant Shigella bacterium and/or the production Shigella bacterium may be a Shigella bacterium in which the virulence plasmid is lost. Loss of the virulence plasmid leads to loss of the msbB2 gene, and the chromosomal msbB1 gene can be inactivated, thereby removing myristoyl transferase activity and providing a penta-acylated lipid A in the LPS. The recombinant Shigella bacterium and/or the production Shigella bacterium may be from S. flexneri msbB mutants lacking the virulence plasmid which contains the msbB2 gene. The recombinant Shigella bacterium and/or the production Shigella bacterium may express penta-acylated LPS.

Alternatively, inactivation of htrB results in loss of the lauroyl chain and thus can yield penta-acylated LPS in some strains and/or forms of lipid A that are less toxic than wild type lipid A. For example, in S. flexneri, inactivation of htrB may be compensated for by the activity of another enzyme, LpxP that results in hexa-acylated lipid A, wherein the lauroyl-chain is replaced by a palmitoleoyl chain. Hexy-acylated lipid A comprising palmitoleoyl chains is less toxic than wild type lipid A. Thus, in some embodiments, the outer membrane vesicles, the recombinant Shigella bacterium and/or the production Shigella bacterium comprise penta-acylated lipid A and or hexa-acylated lipid A wherein the lauroyl-chain is replaced by a palmitoleoyl chain or wherein C14 comprises a myristoyl group. In some embodiments, the outer membrane vesicles, recombinant Shigella bacterium and/or production Shigella bacterium include the following mutations (a) Shigella sonnei: ΔtolR, ΔhtrB, virG::nadAB, (b) Shigella flexneri 2a: ΔtolR, ΔmsbB, (c) Shigella flexneri 3a: ΔtolR, ΔmsbB and (d) Shigella flexneri 6: ΔtolR, ΔmsbB or ΔhtrB. Suitable strains are disclosed in the examples. Other suitable strains are known in the art, for example in WO2011/036564.

### Pharmaceutically Acceptable Excipients, Adjuvants and Immunogenic Compositions

The present invention further provides an immunogenic composition comprising the outer membrane vesicle or recombinant Shigella bacterium of the present invention. The immunogenic compositions of the invention may further comprise a pharmaceutically acceptable carrier. The methods of the invention may comprise a step of formulating the outer membrane vesicles or the recombinant Shigella bacterium comprising at least one heterologous O-antigen with additional components, and the additional components may comprise a pharmaceutically acceptable excipient.

Typical ‘pharmaceutically acceptable excipients’ include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. Pharmaceutically acceptable excipients may also contain diluents, such as water, saline, glycerol, etc. Additionally, auxiliary substances, such as wetting or emulsifying agents, pH buffering substances, and the like, may be present. Sterile pyrogen-free, Tris-buffered physiologic saline is a suitable carrier particularly when using aluminium adjuvants since the phosphate in phosphate buffered saline may interfere with outer membrane vesicle binding to aluminium.

Immunogenic compositions of the invention may include one or more adjuvants. The methods of the invention may comprise a step of formulating the outer membrane vesicles or the recombinant Shigella bacterium comprising at least one heterologous O-antigen with additional components, wherein the additional components comprise an adjuvant.

Particular adjuvants include aluminium adjuvants, for example, aluminium hydroxide (such as ALHYDROGEL®), aluminium phosphate, potassium aluminium sulphate and alum. The use of other adjuvants that also reduce the pyrogenic response is also envisaged and could be identified by the skilled person using the tests exemplified below. Whilst the term “adjuvant” generally refers to any substance that increases the immune response to an antigen, in the present case, and without wishing to be bound by hypotheses, the adjuvant, such as ALHYDROGEL®, is also an adsorbant reducing the immune response to outer membrane vesicles or a Shigella bacterium. Thus, the term “adsorbant” refers to a solid substrate or material to which the outer membrane vesicles or the Shigella bacterium may bind, attach or adsorb (for example, by Van der Waals interactions or hydrogen bonding) such that the pyrogenic response to the outer membrane vesicles or the Shigella bacterium is reduced in comparison to components that are not so bound, attached or adsorbed. By way of non-limiting example, immunogenicity of outer membrane vesicles or a Shigella bacterium may be measured by comparing anti-LPS antibody response.

Immunogenic compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The immunogenic composition may be prepared for topical administration e.g. as an ointment, cream or powder. The immunogenic composition may be prepared for oral administration e.g. as a tablet or capsule, as a spray, or as a syrup (optionally flavoured). The immunogenic composition may be prepared for pulmonary administration e.g. as an inhaler, using a fine powder or a spray. The composition may be prepared as a suppository or pessary. The immunogenic composition may be prepared for nasal, aural or ocular administration e.g. as drops. The immunogenic composition may be in kit form, designed such that a combined composition is reconstituted just prior to administration to a mammal. Such kits may comprise one or more antigens in liquid form and one or more lyophilised antigens. Immunogenic compositions may be presented in vials, or they may be presented in pre-filled syringes. The syringes may be supplied with or without needles. A syringe will include a single dose of the composition, whereas a vial may include a single dose or multiple doses.

Aqueous immunogenic compositions of the invention are also suitable for reconstituting other vaccines from a lyophilised form. Where an immunogenic composition of the invention is to be used for such extemporaneous reconstitution, the invention provides a kit, which may comprise two vials, or may comprise one ready-filled syringe and one vial, with the contents of the syringe being used to reactivate the contents of the vial prior to injection.

Immunogenic compositions of the invention may be packaged in unit dose form or in multiple dose form. For multiple dose forms, vials are preferred to pre-filled syringes. Effective dosage volumes can be routinely established, but a typical human dose of the composition has a volume of 0.5 ml e.g. for intramuscular injection.

The pH of the immunogenic composition is preferably between 6 and 8, for example, 6, 6.5, 7, 7.5 or 8. For compositions comprising acetylated O-antigens particularly the pH of the composition is less than 7, optionally about 6 (to slow the rate of de-esterification).

Stable pH may be maintained by the use of a buffer. The immunogenic compositions of the invention may comprise a Tris [Tris(hydroxymethyl)aminomethane]buffer. The Tris buffer may comprise about 1-20 mM [Tris(hydroxymethyl)aminomethane], e.g. 1.25 mM, 2.5 mM, 5.0 mM or 10.0 mM. For immunogenic compositions comprising acetylated O-antigens, in particular, the buffer is not a Tris buffer. The immunogenic compositions of the invention may comprise a 5-20 mM succinate buffer, e.g. 5 mM, 7.5 mM, 10 mM, 12.5 mM, 15 mM, 17.5 mM or 20 mM. The immunogenic compositions of the invention may comprise a 5-20 mM histidine buffer, e.g. 5 mM, 7.5 mM, 10 mM, 12.5 mM, 15 mM, 17.5 mM or 20 mM. The immunogenic compositions of the invention may comprise a 5-20 mM sodium phosphate buffer e.g. 5 mM, 7.5 mM, 10 mM, 12.5 mM, 15 mM, 17.5 mM or 20 mM. The composition will be sterile. Immunogenic compositions of the invention may be isotonic with respect to humans.

Thus, immunogenic compositions of the invention may be useful as vaccines. Vaccines according to the invention may either be prophylactic (i.e. to prevent infection) or therapeutic (i.e. to treat infection), but will typically be prophylactic.

Immunogenic compositions used as vaccines comprise an immunologically effective amount of antigen(s), as well as any other components, as needed. By “immunologically effective amount”, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

Immunogenic compositions of the invention may include an antimicrobial, particularly when packaged in multiple dose formats.

Immunogenic compositions of the invention may include sodium salts (e.g. sodium chloride) to give tonicity. A concentration of 10±2 mg/ml NaCl is typical. In some embodiments, a concentration of 4 to 10 mg/ml NaCl may be used, e.g. 9.0, 7.0, 6.75 or 4.5 mg/ml.

### Methods of Treatment

The invention also provides a method of preventing or treating Shigella infection in a subject, comprising administering an effective amount of an immunogenic composition or vaccine of the invention to a subject. The invention also provides an immunogenic composition of the invention for use in preventing or treating Shigella infection. The invention also provides the use of an immunogenic composition of the invention in the manufacture of a medicament for preventing or treating Shigella infection.

The term “treating” includes both therapeutic treatment and prophylactic or preventative treatment, wherein the object is to prevent or lessen infection. For example, treating may include directly affecting or curing, suppressing, inhibiting, preventing, reducing the severity of, delaying the onset of, reducing symptoms associated with, for example, infection, or a combination thereof. “Preventing” may refer, inter alia, to delaying the onset of symptoms, preventing relapse of a disease, and the like. “Treating” may also include “suppressing” or “inhibiting” an infection or illness, for example reducing severity, number, incidence or latency of symptoms, ameliorating symptoms, reducing secondary symptoms, reducing secondary infections, prolonging patient survival, or combinations thereof.

The term “preventing or treating Shigella infection” in the method/immunogenic composition for use/use of the immunogenic composition in the manufacture of a medicament of the invention comprises raising an immune response in a subject. The immune response may be protective and may raises antibodies, such as IgG antibodies.

In an embodiment, the method/immunogenic composition for use/use of the immunogenic composition in the manufacture of a medicament of the invention will raise anti-S. sonnei antibodies and/or anti-S. flexneri antibodies. The antibodies may be IgG antibodies. In an embodiment, the method/immunogenic composition for use/use of the immunogenic composition in the manufacture of a medicament of the invention will raise anti-S. flexneri IgG antibodies above a certain antibody titre threshold. The threshold may be between 1:500 and 1:3000, preferably between 1:1000 and 1:2000. The threshold is preferably about 1:1600.

The method/immunogenic composition for use/use of the immunogenic composition in the manufacture of a medicament of the invention may raise a booster response.

The immunogenic composition that is used to prevent or treat Shigella infection may be able to raise an immune response in a subject and may be a vaccine.

The method/immunogenic composition for use/use of the immunogenic composition in the manufacture of a medicament of the invention is optionally for the prevention and/or treatment of illness caused by Shigella e.g. shigellosis, dysentery and associated symptoms including diarrhoea, fever, abdominal pain, tenesmus, etc. These uses and methods are optionally for the prevention and/or treatment of illness caused by both Shigella sonnei and Shigella flexneri, optionally Shigella flexneri 1b, 2a and/or 3a.

The subject of the invention is a mammal, optionally a human. Where the vaccine is for prophylactic use, the human may be an adult i.e. subject is 18 years old or above 18 years old. Where the vaccine is for prophylactic use, the human may be a child i.e. below 18 years old. Where the vaccine is for prophylactic use, the child may be between 12 to 72 months, preferably between 24 to 59 months, more preferably between 6 to 12 months.

Where the vaccine is for prophylactic use, the child may be around 9 months.

Where the vaccine is for therapeutic use, the human is preferably a child.

A vaccine intended for children may also be administered to adults e.g. to assess safety, dosage, or immunogenicity.

Sequence listing table:

| Sequence identity number | Sequence |
|---|---|
| 1 | gtrI |
| 2 | gtrII |
| 3 | gtrIV |
| 4 | gtrV |
| 5 | gtrX |
| 6 | oacA |
| 7 | oacB |
| 8 | oacD |
| 9 | gtrI promoter |
| 10 | gtrII promoter |
| 11 | gtrIV promoter |
| 12 | gtrV promoter |
| 13 | gtrX promoter |
| 14 | oacA promoter |
| 15 | oacB promoter |
| 16 | oacD promoter |

### Example 1—General Methods

### Bacterial Strains and Growth Condition

All Shigella flexneri strains were obtained from Public Health England (Table 3). All bacterial strains and derivative mutants were grown at 30° C. in liquid Luria-Bertani (LB) medium, supplemented with the appropriate antibiotic, in a rotary shaker for 16 hours. For GMMA production, overnight cultures were diluted in HTMC medium (15 g/L Glycerol, 30 g/L Yeast extract, 0.5 g/L MgSO₄, 5 g/L KH₂PO₄, 20 g/L K₂HPO₄), supplemented with the appropriate antibiotic, to an optical density at 600 nm (OD600) of 0.3 and grown at 30° C. in a rotary shaker for 8 hours using baffled flasks with a liquid to air volume ratio of 1:5.

### FACS Analysis

S. flexneri strains were grown overnight at 30° C. in LB supplemented with the appropriate antibiotic. Bacteria were then pelleted and washed with PBS at 8,000×g for 5 minutes. Bacteria were then blocked with PBS containing 3% (w/v) Bovine Serum Albumin (BSA) for 15 minutes and incubated with polyclonal rabbit sera [Denka Saiken] diluted in PBS+1% (w/v) BSA (1:500) for 1 hour. After washes, samples were incubated with Alexa Fluor 488 mouse anti-rabbit IgG (1:500) [Molecular Probes] for 30 minutes. Finally, bacteria were fixed with 4% (w/v) formaldehyde for 20 minutes and flow cytometry analysis was performed using FACS Canto 11 flow cytometer [BD Biosciences].

### GMMA Production and Characterization

After growth in HTMC medium, bacteria were pelleted through centrifugation at 5,000×g for 45 minutes. Cell-free supernatants were recovered and filtered through 0.22 μm Stericup filters [Millipore]. After ultracentrifugation of filtered supernatants at 175,000×g for 2 hours at 4° C., the resulting pellet, containing GMMA, was washed with PBS, further ultracentrifuged at 175,000×g for 2 hours and finally resuspended in PBS. GMMA purity was assessed by HPLC-SEC analysis; total protein content was estimated by micro BCA using bovine serum albumin (BSA) as a reference following the manufacturer's instructions [Thermo Scientific]; OAg sugar content was quantified by HPAEC-PAD, as previously described in De Benedetto, G. et al. Characterization of O-antigen delivered by Generalized Modules for Membrane Antigens (GMMA) vaccine candidates against nontyphoidal Salmonella. Vaccine 35, 419-426, doi:10.1016/j.vaccine.2016.11.089 (2017) and Micoli, F. et al. Structural analysis of O-polysaccharide chains extracted from different Salmonella Typhimurium strains. Carbohydr Res 385, 1-8, doi:10.1016/j.carres.2013.12.003 (2014).

### Immunogenicity Studies in Mice

S. flexneri GMMA and their combinations were tested in mice. Animal studies were performed at Toscana Life Science Animal Care Facility under the animal project 479/2017-PR Sep. 6, 2017 approved by the Italian Ministry of Health. Five weeks old female CD1 mice (8 per group) were immunized intraperitoneally with 200 L of vaccine at day 0 and 28. Doses were normalized so that each group received 0.5 μg of serotype-specific OAg. Single sera collected 2 weeks after second injection were tested for SBA against a panel of S. flexneri strains using a high throughput assay based on luminescent readout (Necchi, F., Saul, A. & Rondini, S. Setup of luminescence-based serum bactericidal assay against Salmonella Paratyphi A. J Immunol Methods 461, 117-121, doi:10.1016/j.jim.2018.06.025 (2018)). Results of the assay were expressed as the IC50, the reciprocal serum dilution that resulted in a 50% reduction of luminescence and thus corresponding to 50% growth inhibition of the bacteria present in the assay. GraphPad Prism 7 software was used for curve fitting and IC50 determination. Titers below the minimum measurable signal were assigned a value of 50, corresponding to half of the first dilution of sera tested.

### OAg Purification and Characterization

OAg extraction and purification from GMMA was performed as previously described (De Benedetto, G. et al. Characterization of O-antigen delivered by Generalized Modules for Membrane Antigens (GMMA) vaccine candidates against nontyphoidal Salmonella. Vaccine 35, 419-426, doi:10.1016/j.vaccine.2016.11.089 (2017); Raso, M. M. et al. GMMA and Glycoconjugate Approaches Compared in Mice for the Development of a Vaccine against Shigella flexneri Serotype 6. Vaccines (Basel) 8, doi:10.3390/vaccines8020160 (2020); and Micoli, F. et al. A scalable method for O-antigen purification applied to various Salmonella serovars. Anal Biochem 434, 136-145, doi:10.1016/j.ab.2012.10.038 (2013). OAg populations were characterized by HPLC-SEC with differential refractive index (dRI) detection to estimate the molecular size distribution. OAg peak molecular weight (MP) was calculated using dextrans as standards in the range 12-150 kDa. Sugar content was quantified by HPAEC-PAD, after removal of the low molecular weight core molecules by filtration through Amikon 10k. The linkage positions for the constituent sugars of the OAg samples were determined by GLC-MS of the partially methylated alditol acetate (PMAA) derivatives. ¹H-NMR spectroscopy was used to confirm OAg identity and structure. All NMR experiments were performed with a Bruker AEON AVANCE III 600 MHz spectrometer equipped with a high-precision temperature controller using a 5 mm QCI CryoProbe®. NMR spectra were recorded at 50.0±0.1° C. The transmitter was set at the water frequency (4.70 ppm). Proton spectra were acquired using a 90-degree pulse duration automatically calculated and collected with 32K data points over a 12 ppm spectral width, accumulating 128 number of scans. Spectra were processed by applying an exponential function to the FID with a line broadening of 0.80 Hz to increase the signal-to-noise ratio and then Fourier transformed. Data acquisition and processing were performed with TopSpin 3.5 software package [Bruker BioSpin].

### Example 2—Generation and Complementation of a S. flexneri Scaffold Strain

At first, we generated a GMMA-producing strain by removing the tolR gene in S. flexneri serotype 2a. To generate the GMMA-producing mutants, the kanamycin resistance gene aph was used to replace the tolR gene. The resistance cassette replacement construct was amplified from the pKD4 vector using forward and reverse primers composed of 50 bp homologous to the flanking regions of the gene to be deleted and approximately 20 bp (Table S3) at the 3′ end matching the flanking region of the resistance gene (discussed in Datsenko, K. A. & Wanner, B. L. One-step inactivation of chromosomal genes in Escherichia coli K-12 using PCR products. Proc Natl Acad Sci USA 97, 6640-6645, doi:10.1073/pnas.120163297 (2000); referred to herein as Datsenko and Wanner (2000). PCR products were purified and were used to transform recombination-prone S. flexneri recipient cells carrying pKD46, following methods previously described (Datsenko and Wanner (2000)). Following the tolR gene deletion, the kanamycin resistance gene was removed through FLP-mediated recombination using the pCP20 plasmid to yield markerless mutant strains (Datsenko and Wanner (2000)).

Then, we converted S. flexneri serotype 2a ΔtolR into a serotype Y scaffold strain by removing the phage genes responsible for O-Acetylation of RhaIII (oacB gene), glucosylation of RhaI (gtrII gene) and O-Acetylation of GlcNAc (oacD gene) Table 1. To generate the GMMA-producing mutants, the kanamycin resistance gene aph was used to replace the tolR gene. The resistance cassette replacement construct was amplified from the pKD4 vector using forward and reverse primers composed of 50 bp homologous to the flanking regions of the gene to be deleted and approximately 20 bp (see Table 4 at the end of the Examples section for a list of the primers used) at the 3′ end matching the flanking region of the resistance gene (Datsenko and Wanner (2000)). PCR products were purified and were used to transform recombination-prone S. flexneri recipient cells carrying pKD46, following methods previously described (Datsenko and Wanner (2000)). Following the tolR gene deletion, the kanamycin resistance gene was removed through FLP-mediated recombination using the pCP20 plasmid to yield markerless mutant strains (Datsenko and Wanner (2000)).

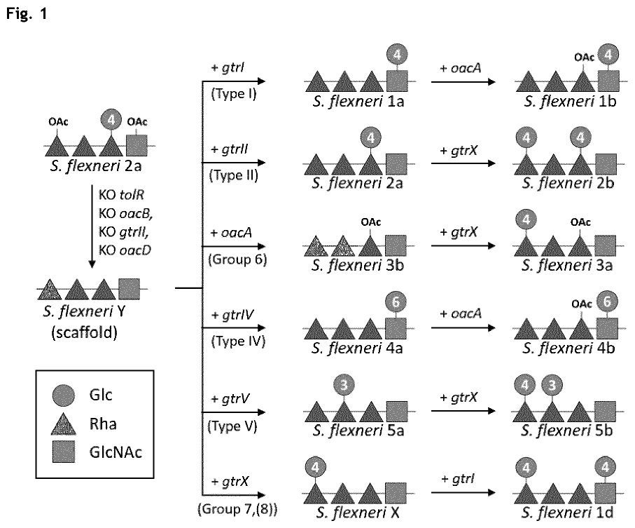

GMMA resulting from the scaffold strain were fully characterized with a panel of analytical methods (described in the section entitled “OAg purification and characterization”) to confirm the display of the expected OAg (data set out in Table 2 at the end of the examples section,). In addition to the data shown in Table 2, NMR spectra of the scaffold strain were generated (as described in the section entitled “OAg purification and characterization”) and showed that the scaffold strain lacked O-Acetylation of RhaIII glucosylation of RhaI and 0-Acetylation of GlcNAc. Next, we generated a set of plasmids based on the pCOLA-Duet and pACYC-Duet systems for the complementation of five known glucosyltransferases (gtrI, gtrII, gtrIV, gtrV, gtrX) and one O-Acetyltransferase (oacA) responsible for the majority of the OAg modifications in S. flexneri (Table 1, Table 3). All genes were cloned together with their native promoters to be constitutively expressed and the plasmids were selected in the presence of kanamycin (pCOLA-Duet) or chloramphenicol (pACYC-Duet). Transformation of the S. flexneri serotype Y scaffold strain with the 6 plasmids and their combinations yielded 12 converted strains (FIG. 1, Table 3). FACS typing analysis on the bacterial cells and Nuclear Magnetic Resonance (NMR) (carried out as described above in the sections entitled “OAg purification and characterization” and “FACS analysis”) on the OAg extracted from the corresponding GMMA confirmed all the expected OAg structures resulting from the conversion of serotype Y to all the known S. flexneri serotypes (1a, 1b, 1d, 2a, 2b, 3a, 3b, 4a, 4b, 5a, 5b, X) (Table 6).

### Example 3—Complementation of Naturally Non-Occurring Combinations of OAg-Modifying Enzymes

While certain combinations of OAg-modifying enzymes do not occur in nature due to bacteriophages incompatibility in infecting the same bacterium, our strategy allowed for the first time to complement S. flexneri with naturally non-occurring combinations of glucosyl- and O-Acetyl-transferases. Complementation of the scaffold strain with all the possible combinations of two OAg-modifying enzymes which naturally do not occur together yielded 9 previously undescribed serotypes displaying multiple type and group epitopes at the same time, as verified by FACS typing analysis (Table 6).

To increase the number of co-expressed OAg-modifying enzymes and push the approach even further, we also complemented a S. flexneri serotype 3a GMMA-producing strain, which naturally carries the gtrX and oacA genes (Table 1), with the previously described plasmids yielding 7 additional strains displaying up to five type factors and two group factors at the same time (Table 7).

From the FACS analysis, it was clear that all bacterial populations were uniformly expressing the complemented enzymes and therefore displaying the corresponding type and group factors (not shown). Nevertheless, it was critical to understand whether the OAg chains were constituted by (a) different RU, each carrying some of the complemented type and group factors or (b) identical RU modified by all the complemented OAg-modifying enzymes. To discriminate between the two scenarios envisaged, the OAg extracted from GMMA purified from all the strains obtained were fully characterized through ¹H-NMR (as described above in the section entitled “Oag purification and characterization”).

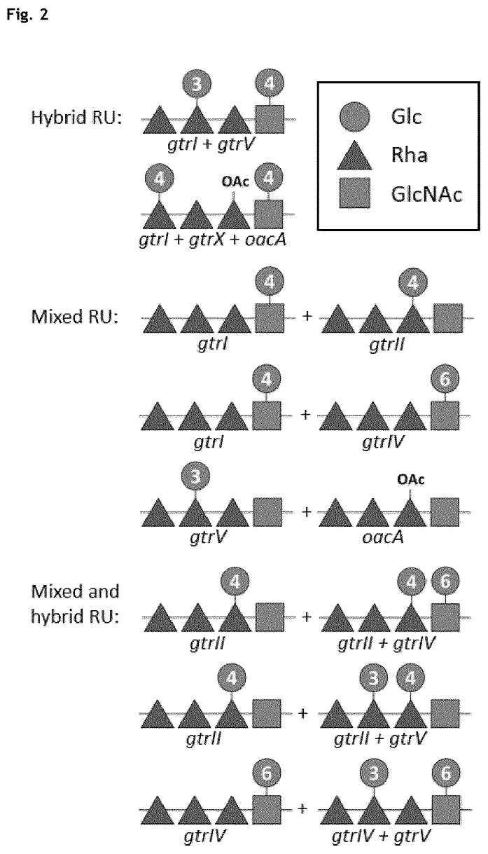

Interestingly, only one combination of two OAg-modifying enzymes (gtrI+gtrV) and one combination of three OAg-modifying enzymes (gtrI+gtrX+oacA) were able to modify 100% of the RU (hybrid RU, FIG. 2, Table 8), similarly to what observed with naturally occurring enzyme combinations. All other combinations of enzymes yielded mixed OAg chains, indicating that certain enzymes are not able to modify the sugar repeat once it has been previously modified by a different enzyme. In particular, ¹H-NMR showed that certain combinations of enzymes resulted in OAg chains composed by two different RU corresponding to individual serotypes combined in a 1:1 ratio (i.e. gtrI+gtrII, gtrI+gtrIV, gtrV+oacA) (mixed RU, FIG. 2, Table 8). Other combinations resulted in OAg chains composed of two different RU, only one of which was modified by both enzymes (i.e. gtrII+gtrIV, gtrII+gtrV, gtrIV+gtrV) (mixed and hybrid RU, FIG. 2, Table 8).

### Example 4—Comparison of GMMA from the Converted Scaffold Strains to GMMA from the Corresponding Natural Strains

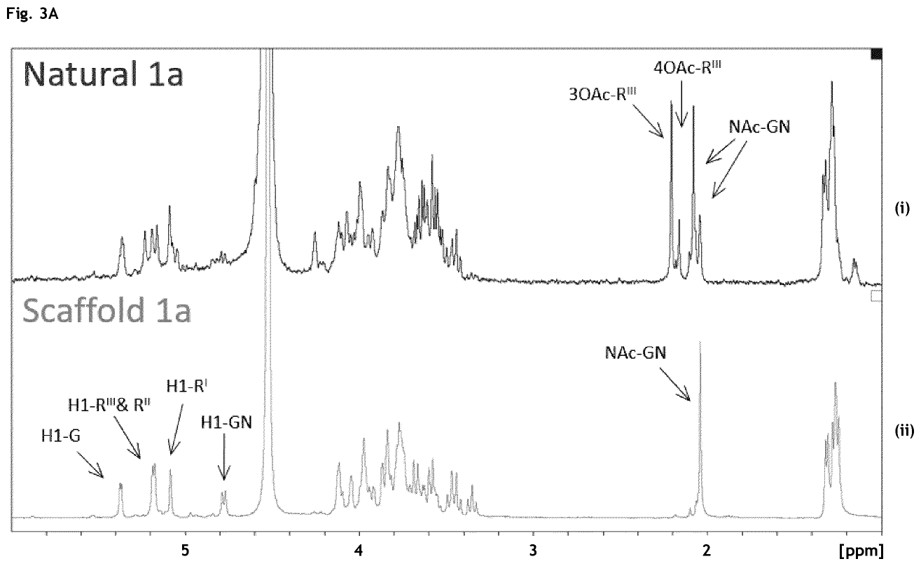

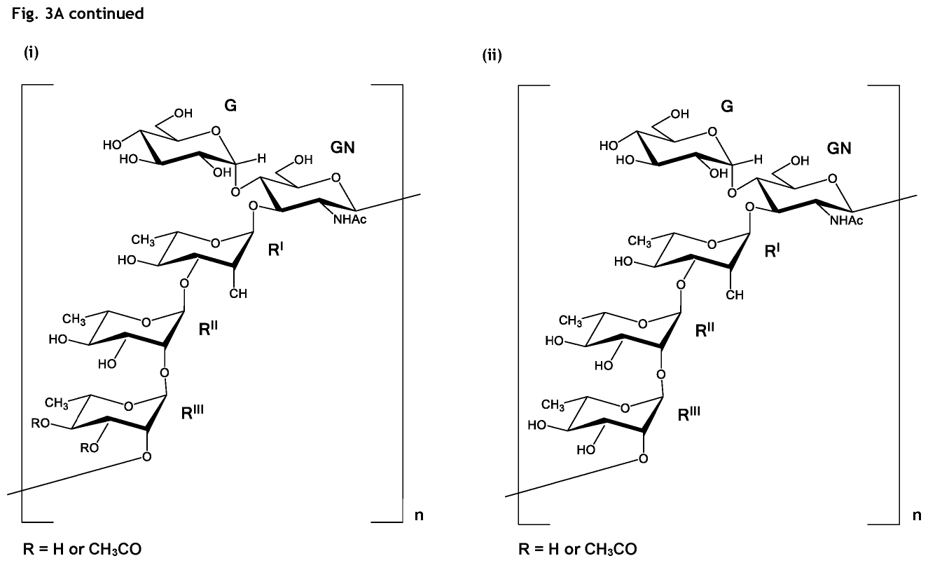

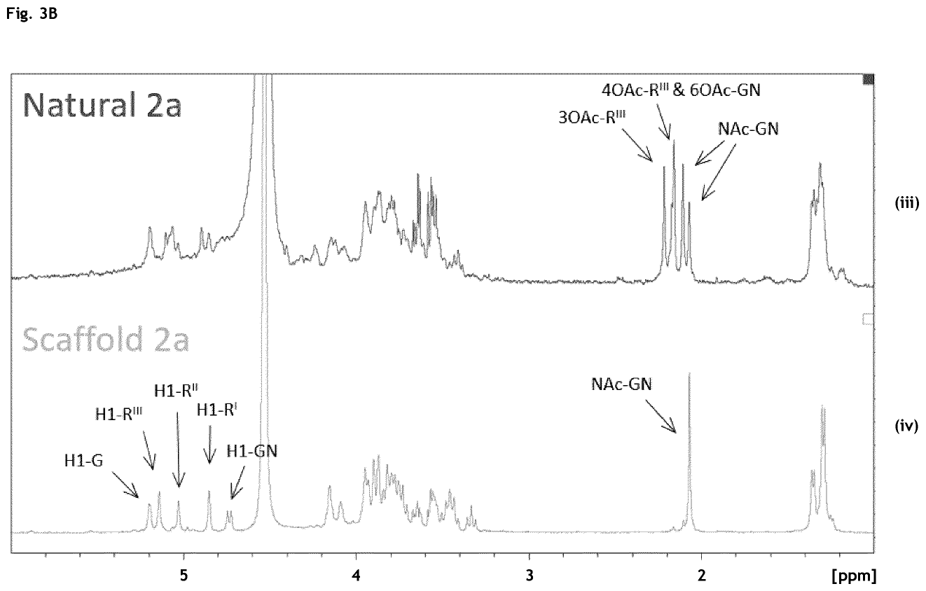

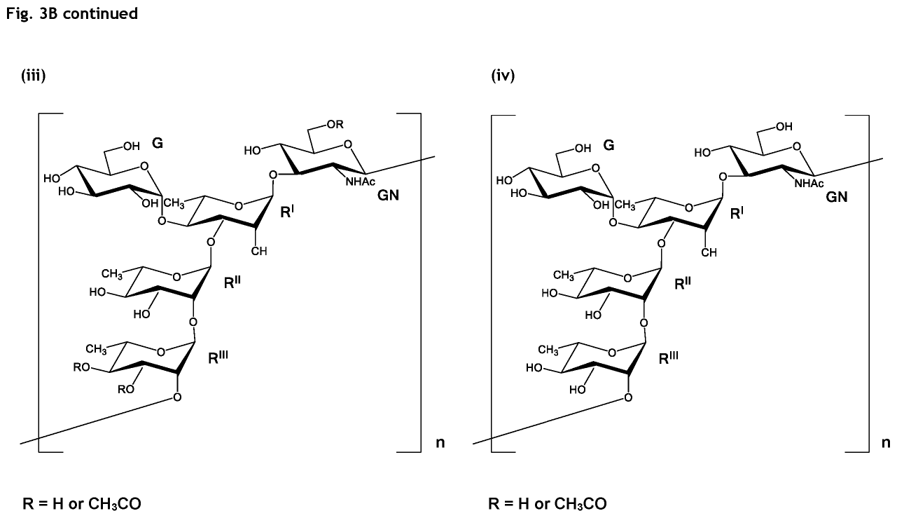

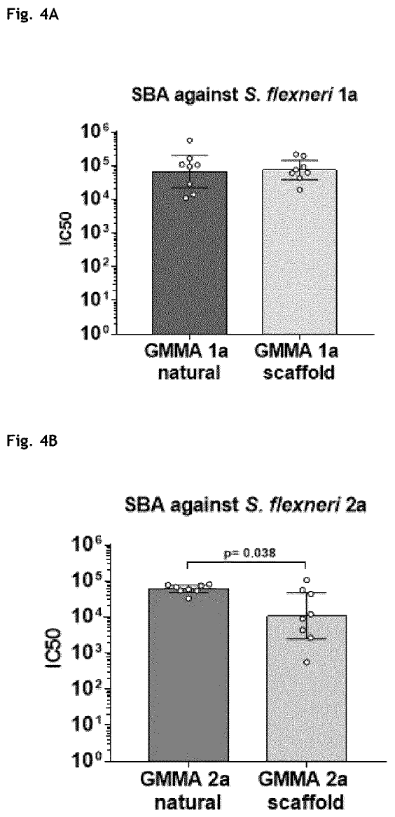

GMMA from the scaffold strain converted to serotype 1a and 2a were fully characterized in comparison to GMMA from natural S. flexneri serotype 1a and 2a (FIG. 3, Table 2). The only differences observed were related to the presence of O-Acetyl groups in GMMA obtained from natural serotypes (OAc-RhaII in serotype 1a, OAc-RhaIII and OAc-GlcNAc in serotype 2a) which were not complemented in the scaffold strains (FIG. 3) and the lack of high molecular weight (HMW) OAg in GMMA obtained from natural serotype 1a (Table 2). When tested in mice at the same OAg dose, GMMA derived from the converted scaffold strain were able to induce antibodies with functional activity, similarly to the corresponding GMMA derived from natural strains (FIG. 4). In particular, no difference was observed between natural 1a and scaffold 1a GMMA in the elicited serum bactericidal activity against S. flexneri 1a, while a slightly lower response was measured with scaffold 2a GMMA compared to natural 2a GMMA in the elicited serum bactericidal activity against S. flexneri serotype 2a (p=0.038) (FIG. 4).

### Example 5—Immunogenicity of GMMA Displaying Mixed or Hybrid Serotypes Compared to Physical Mixtures of GMMA Displaying the Corresponding Individual Serotypes

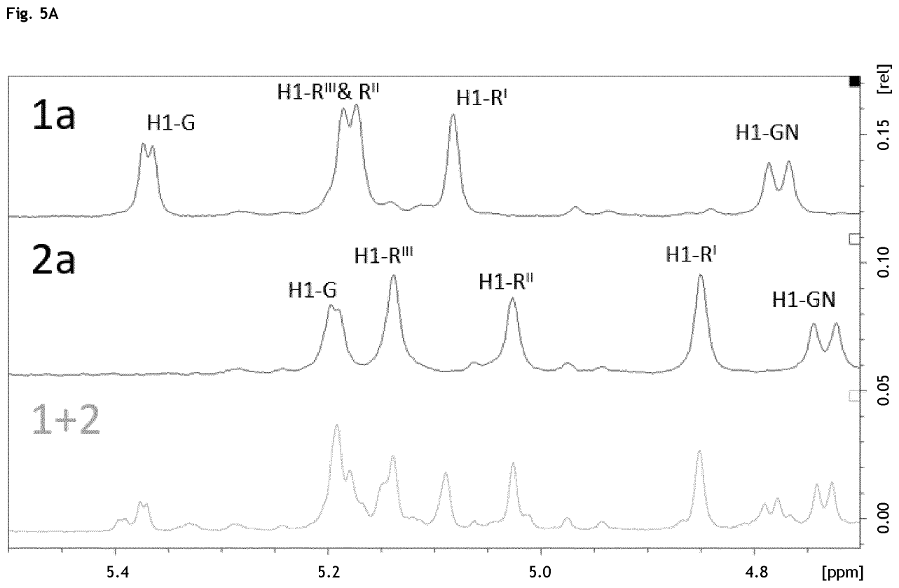

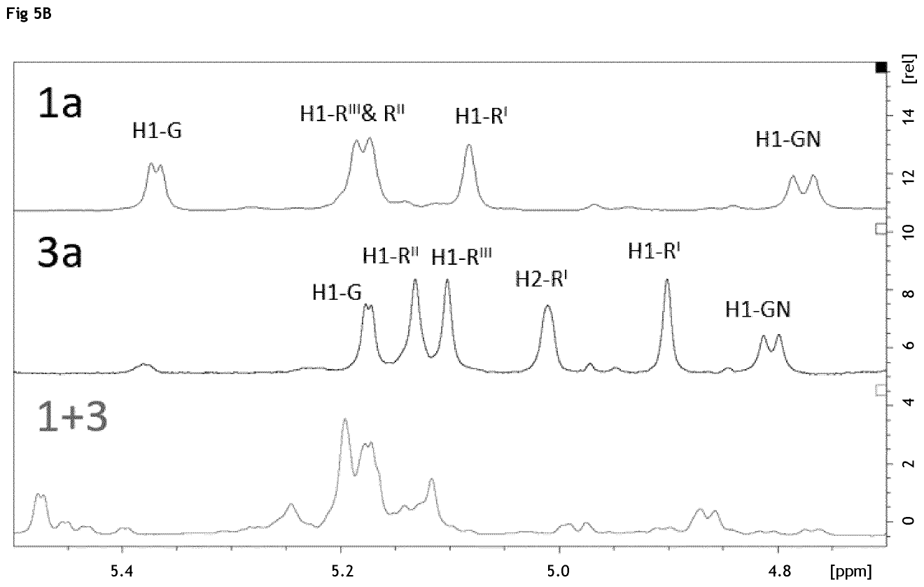

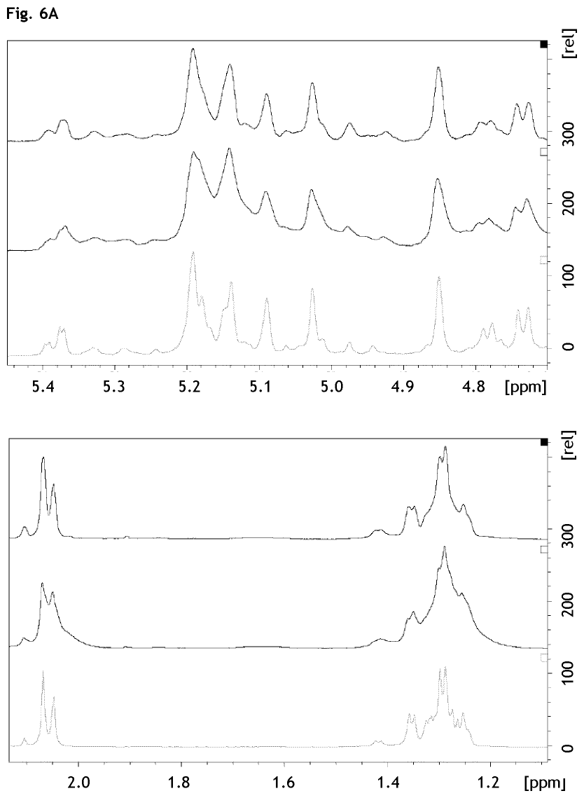

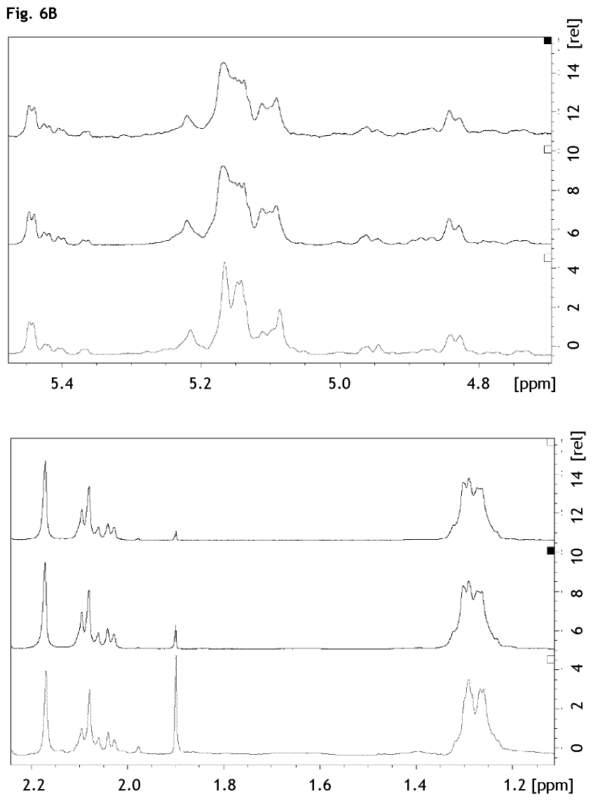

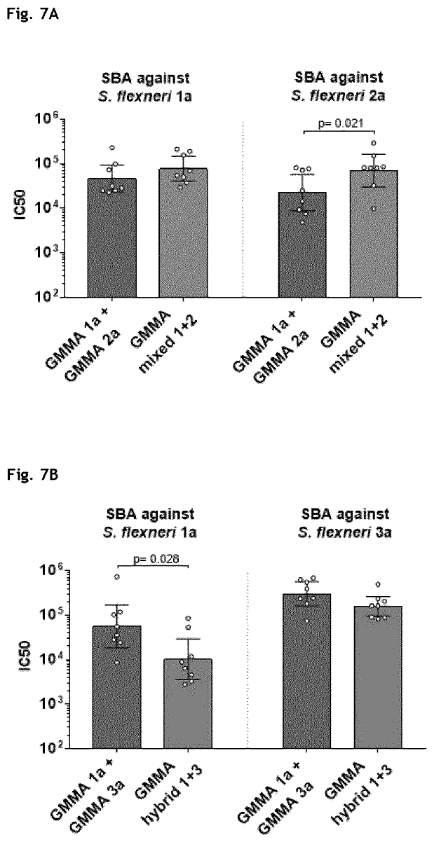

Representatives of GMMA displaying mixed or hybrid OAg were selected to investigate their immunogenicity in mice. GMMA from the mixed serotype 1+2 (gtrI+gtrII) and the hybrid serotype 1+3 (gtrI+gtrII+oacA) were fully characterized through High Performance Anion Exchange Chromatography-Pulsed Amperometric Detection (HPAEC-PAD) analysis, Gas-Liquid Chromatography-Mass Spectrometry (GLC-MS) and ¹H-NMR (as described in the section entitled “OAg purification and characterization” above) (FIG. 5, Table 2, Table 5). GMMA from the mixed serotype 1+2 were characterized by OAg chains composed by a mixed population of RU, 50% of which being glucosylated on GlcNAc (as in serotype 1a) and 50% on RhaI (as in serotype 2a). On the other hand, GMMA from the hybrid serotype 1+3 were characterized by OAg chains composed by RU glucosylated both on RhaIII and on GlcNAc (as in serotype 1d) and O-Acetylated on RhaI (similarly to serotype 3a and 3b). Importantly, lot to lot consistency of these structural features were verified on 3 different GMMA preparations (FIG. 6). Both GMMA were tested in mice to compare their immunogenicity to that of physical mixtures of GMMA displaying the corresponding individual serotypes, normalizing the doses so that every group received the same amount of each serotype-specific OAg. GMMA carrying mixed or hybrid OAg induced functional antibodies against both the displayed serotypes (FIG. 7). In particular, GMMA displaying the mixed 1+2 serotype elicited higher Serum Bactericidal Activity (SBA) titers against S. flexneri 2a compared to the physical mixture of scaffold 1a and 2a GMMA (p=0.021). On the contrary, GMMA displaying the hybrid 1+3 serotype elicited lower SBA titers against S. flexneri 1a compared to the physical mixture of scaffold 1a GMMA and natural 3a GMMA (p=0.028).

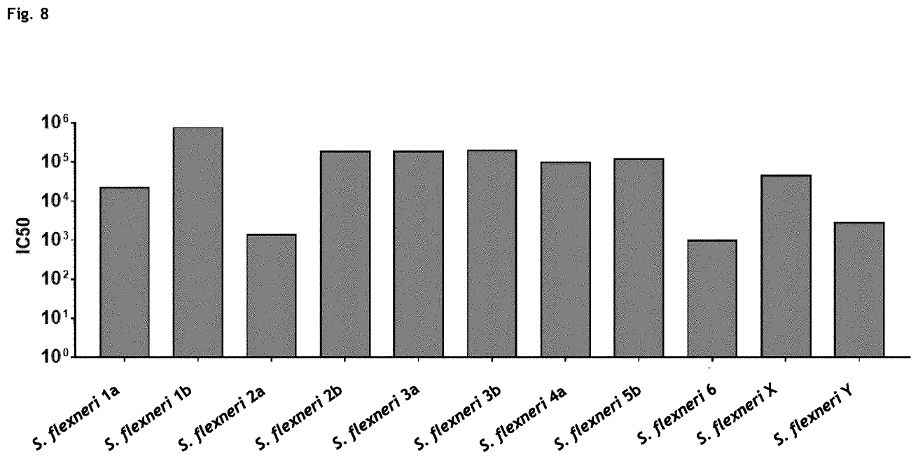

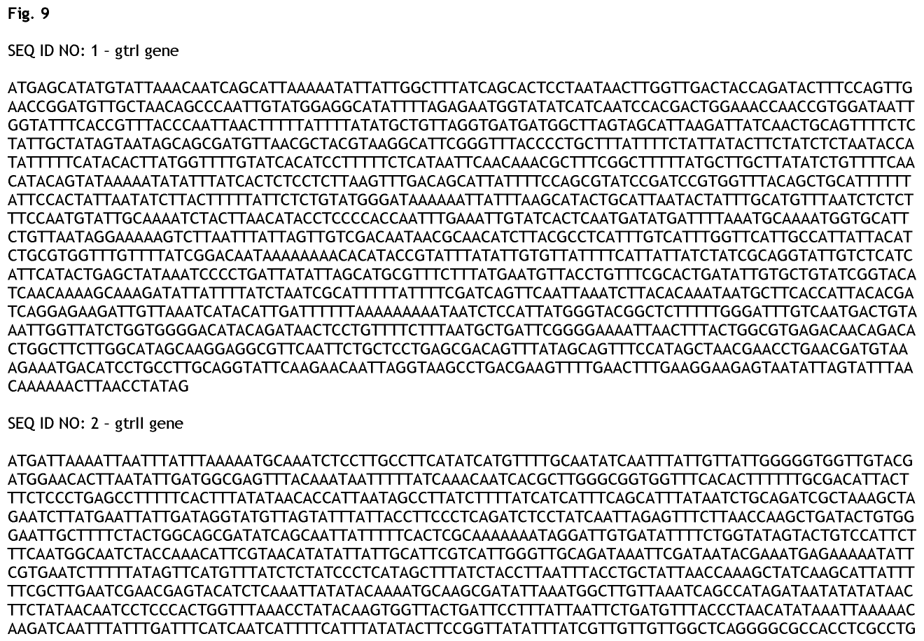

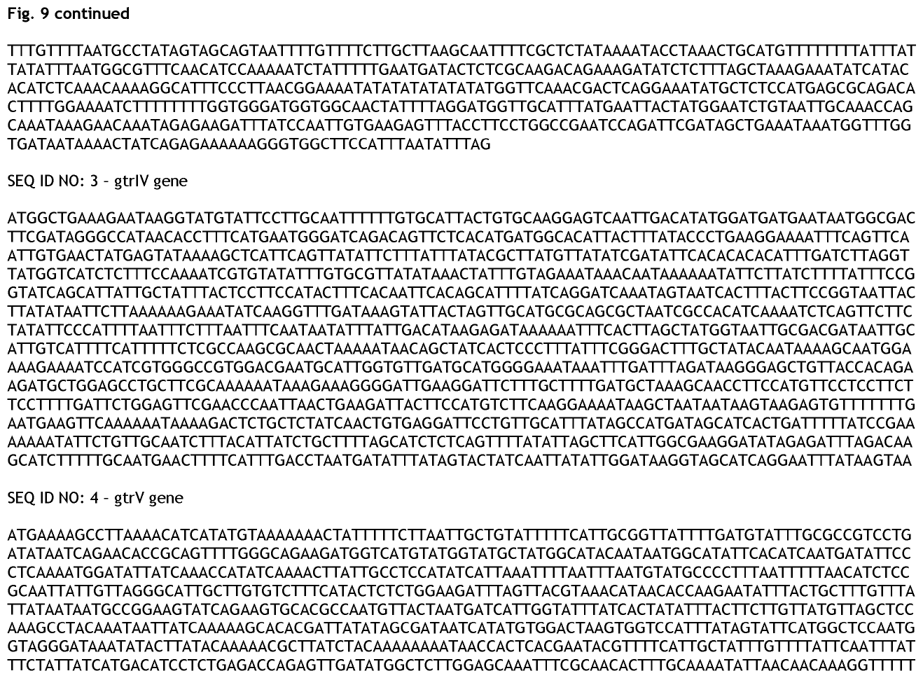

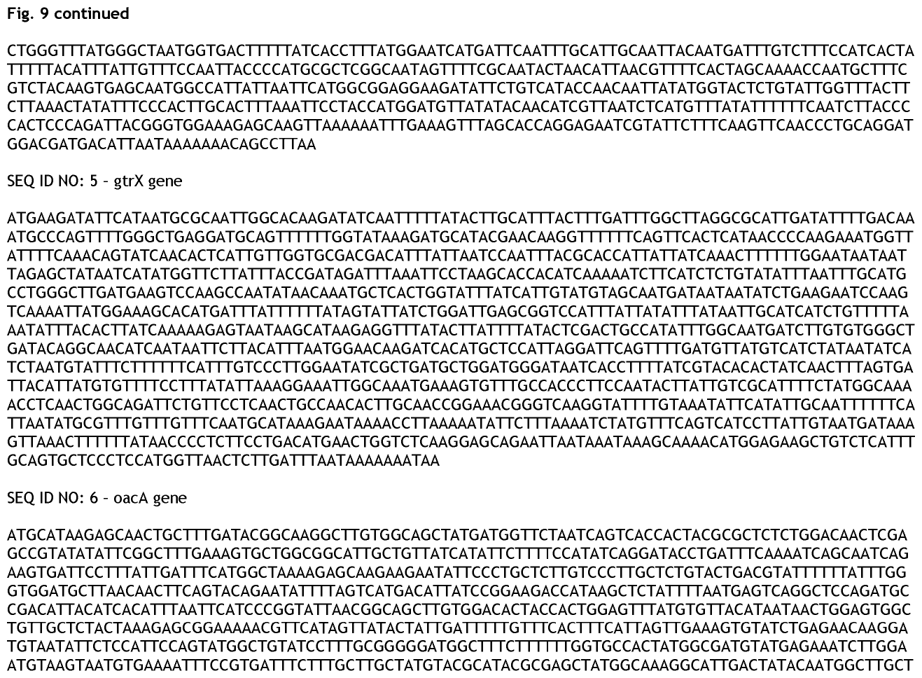

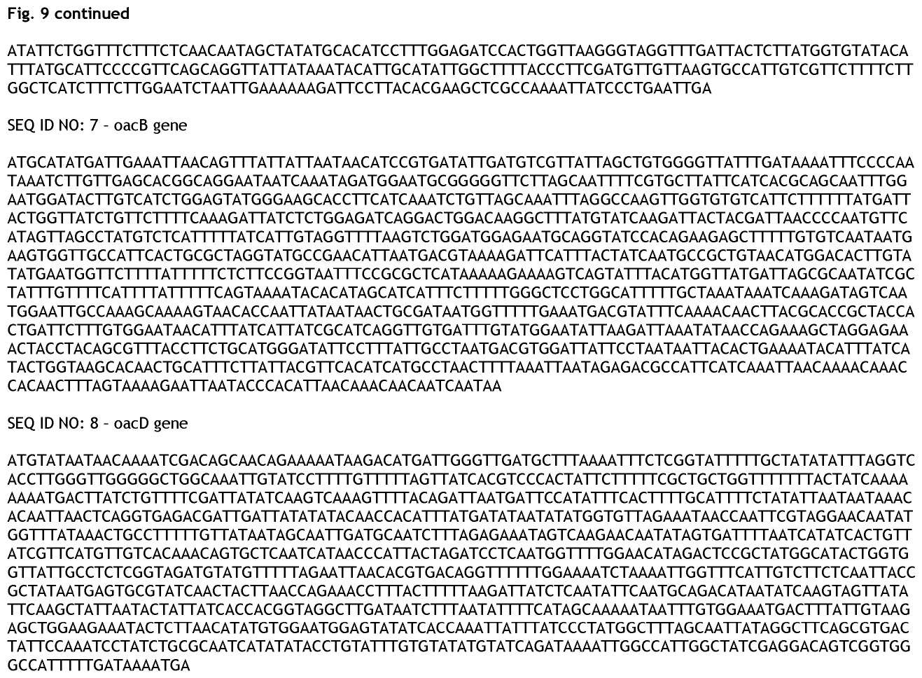

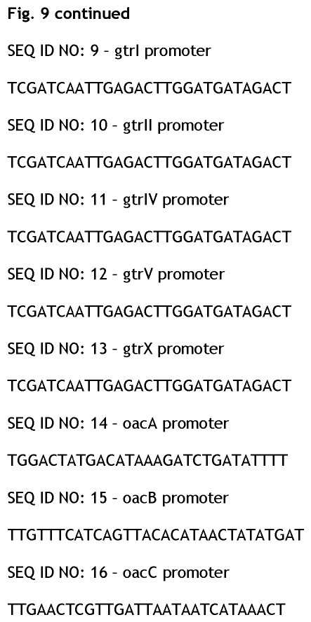

Based on previously collected cross-reactivity data in mice and considering the serotype specificities displayed on GMMA from the hybrid 1+3 serotype we asked whether such GMMA could induce broadly cross-reactive antibodies. Importantly, SBA analysis of the sera from mice immunized with GMMA obtained from the hybrid 1+3 serotype confirmed their ability to induce killing of all the most epidemiologically relevant S. flexneri serotypes (FIG. 8).

TABLE 2
Analytical characterization of the GMMA used in the immunogenicity

| | | OAg/ | | | Rha/Glc |
|---|---|---|---|---|---|
| | | protein | OAg size | | ratio |
| | | ratio | (HPLC-SEC dRI) | | (HPAEC- |
| GMMA ID: | GMMA from strain: | (w/w) | kDa | Area | PAD) |
| GMMA Y | S. flexneri scaffold | 0.54 | 77 | 16% | NA |
| scaffold | ΔtolR::frt | | 17 | 48% | |
| | | | 2 | 36% | |
| GMMA 1a | S. flexneri scaffold | 0.49 | 58 | 20% | 2.4 |
| scaffold | ΔtolR::frt | | 14 | 43% | |
| | pCOLA-Duet_gtrI | | 2 | 37% | |
| GMMA 2a | S. flexneri scaffold | 0.73 | 59 | 22% | 2.2 |
| scaffold | ΔtolR::frt | | 14 | 43% | |
| | pCOLA-Duet_gtrII | | 2 | 35% | |
| GMMA | S. flexneri scaffold | 0.50 | 55 | 20% | 2.4 |
| mixed 1 + 2 | ΔtolR::frt | | 14 | 43% | |
| | pCOLA-Duet_gtrI | | 2 | 37% | |
| | pACYC-Duet_gtrII | | | | |
| GMMA | S. flexneri 3a | 0.65 | 64 | 26% | 1.5 |
| hybrid 1 + 3 | ΔtolR::frt | | 16 | 46% | |
| | pCOLA-Duet_gtrI | | 2.5 | 28% | |
| GMMA 1a | S. flexneri 1a | 0.60 | 13 | 59% | 2.6 |
| natural | ΔtolR::aph | | 2 | 41% | |
| GMMA 2a | S. flexneri 2a | 0.40 | 67 | 15% | 2.4 |
| natural | ΔtolR::frt | | 14 | 50% | |
| | | | 2 | 35% | |
| GMMA 3a | S. flexneri 3a | 0.65 | 71 | 22% | 2.5 |
| natural | ΔtolR::frt | | | | |

TABLE 3
List of all the plasmids and strains used in the Examples

| Complementation plasmids | Reference |
|---|---|
| pKD46 | Datskenko et al., |
| pKD4 | 2000 |
| pCP20 | |
| pCOLA-Duet | Novagen |
| pACYC-Duet | Novagen |
| pCOLA-Duet_gtrI | This study |
| pCOLA-Duet_gtrII | This study |
| pCOLA-Duet_gtrIV | This study |
| pCOLA-Duet_gtrV | This study |
| pCOLA-Duet_gtrX | This study |
| pCOLA-Duet_oacA | This study |
| pACYC-Duet_gtrI | This study |
| pACYC-Duet_gtrII | This study |
| pACYC-Duet_gtrIV | This study |
| pACYC-Duet_gtrV | This study |
| pACYC-Duet_gtrX | This study |
| pACYC-Duet_oacA | This study |
| pCOLA-Duet_gtrI + gtrII | This study |
| pACYC-Duet_gtrIV + gtrV | This study |

| Strains | Reference |
|---|---|
| S. flexneri serotype 1a isolate H130920139 | PHE |
| S. flexneri serotype 1b isolate H130920140 | PHE |
| S. flexneri serotype 2a isolate H130920142 | PHE |
| S. flexneri serotype 2b isolate H130920143 | PHE |
| S. flexneri serotype 3a isolate H130920144 | PHE |
| S. flexneri serotype 3b isolate H130920145 | PHE |
| S. flexneri serotype 4a isolate H130920147 | PHE |
| S. flexneri serotype 4b isolate H130920148 | PHE |
| S. flexneri serotype 5a isolate H130920150 | PHE |
| S. flexneri serotype 5b isolate H130920151 | PHE |
| S. flexneri serotype 6 isolate H130920152 | PHE |
| S. flexneri serotype X isolate H130920153 | PHE |
| S. flexneri serotype Y isolate H130920154 | PHE |
| S. flexneri 1a ΔtolR::aph | This study |
| S. flexneri 2a ΔtolR::frt | This study |
| S. flexneri 3a ΔtolR::frt | This study |
| S. flexneri 2a ΔtolR::frt ΔoacD-gtrII-oacB::frt | This study |
| (“S. flexneri scaffold”) | |
| S. flexneri scaffold ΔtolR::frt pCOLA-Duet_gtrI | This study |
| S. flexneri scaffold ΔtolR::frt pCOLA-Duet_gtrII | This study |
| S. flexneri scaffold ΔtolR::frt pCOLA-Duet_gtrIV | This study |
| S. flexneri scaffold ΔtolR::frt pCOLA-Duet_gtrV | This study |
| S. flexneri scaffold ΔtolR::frt pCOLA-Duet_gtrX | This study |
| S. flexneri scaffold ΔtolR::frt pCOLA-Duet_oacA | This study |
| S. flexneri scaffold ΔtolR::frt pCOLA-Duet_gtrI | This study |
| pACYC-Duet_oacA | |
| S. flexneri scaffold ΔtolR::frt pCOLA-Duet_gtrI | This study |
| pACYC-Duet_gtrX | |
| S. flexneri scaffold ΔtolR::frt pCOLA-Duet_gtrII | This study |
| pACYC_Duet_gtrX | |
| S. flexneri scaffold ΔtolR::frt pCOLA-Duet_gtrX | This study |
| pACYC-Duet_oacA | |
| S. flexneri scaffold ΔtolR::frt pCOLA-Duet_gtrIV | This study |
| pACYC-Duet_oacA | |
| S. flexneri scaffold ΔtolR::frt pCOLA-Duet_gtrX | This study |
| pACYC-Duet_gtrV | |
| S. flexneri scaffold ΔtolR::frt pCOLA-Duet_gtrI | This study |
| pACYC-Duet_gtrII | |
| S. flexneri scaffold ΔtolR::frt pCOLA-Duet_gtrIV | This study |
| pACYC-Duet_gtrI | |
| S. flexneri scaffold ΔtolR::frt pCOLA-Duet_gtrI | This study |
| pACYC-Duet_gtrV | |
| S. flexneri scaffold ΔtolR::frt pCOLA-Duet_gtrII | This study |
| pACYC-Duet_oacA | |
| S. flexneri scaffold ΔtolR::frt pCOLA-Duet_gtrIV | This study |
| pACYC-Duet_gtrII | |
| S. flexneri scaffold ΔtolR::frt pCOLA-Duet_gtrII | This study |
| pACYC-Duet_gtrV | |
| S. flexneri scaffold ΔtolR::frt pCOLA-Duet_gtrV | This study |
| pACYC-Duet_oacA | |
| S. flexneri scaffold ΔtolR::frt pCOLA-Duet_gtrIV | This study |
| pACYC-Duet_gtrV | |
| S. flexneri scaffold ΔtolR::frt pCOLA-Duet_gtrIV | This study |
| pACYC-Duet_gtrX | |
| S. flexneri 3a ΔtolR::frt pCOLA-Duet_gtrI | This study |
| S. flexneri 3a ΔtolR::frt pCOLA-Duet_gtrII | This study |
| S. flexneri 3a ΔtolR::frt pCOLA-Duet_gtrIV | This study |
| S. flexneri 3a ΔtolR::frt pCOLA-Duet_gtrV | This study |
| S. flexneri 3a ΔtolR::frt pCOLA-Duet_gtrI + gtrII | This study |
| S. flexneri 3a ΔtolR::frt pACYC-Duet_gtrIV + gtrV | This study |
| S. flexneri 3a ΔtolR::frt pCOLA-Duet_gtrI + gtrII | This study |
| pACYC-Duet_gtrIV + gtrV | |

TABLE 4
List of all the primers used in the Examples

| | | Restriction | DNA |
|---|---|---|---|
| Primer name | 5′-3′ Sequence | enzyme | template |
| tolR KO_F | accgccaggcgtttaccgttagcgagagcaacaaggggtaagccatggcc | — | pKD4 |
| | GTGTAGGCTGGAGCTGCTTC | | |
| tolR KO_R | acccgctctctttcaagcaagggaaacgcagatgtttagataggctgcgt | — | |
| | CATATGAATATCCTCCTTAG | | |
| oacD-gtrII-oacB | ttttatctgatacatatacacaaatacaggtatatatgattgcgcagata | — | pKD4 |
| KO_F | GTCTTGAGCGATTGTGTAGG | | |
| oacD-gtrII-oacB | tggtttgttttgttaatttgatgaatggcgtctctattaatttaaaagtt | — | |
| KO_R | TCCTCCTTAGTTCCTATTCC | | |
| gtrI_F | cgcggatccAAGCCTTGCAAGCCATTGTG | BamHI | S. flexneri 1a |
| gtrI_R | catgccatggCCCGTCGAAAAAGACGGGTT | NcoI | |
| gtrII_F | ataagaatgcggccgcAAGCCTTGCAAGCCATTGTG | NotI | S. flexneri 2a |
| gtrII_R | cccaagcttGAGCTTGAAAAAGGGAGGCG | HindIII | |
| gtrIV_F | ataagaatgcggccgcTGTCACAAATACGGCACAACG | NotI | S. flexneri 4a |
| gtrIV_R | cccaagcttAGAGCAAGATTTAACGCTACATAAA | HindIII | |
| gtrV_F | cggggtaccACACGTCCCACCACATCAAA | KpnI | S. flexneri 5a |
| gtrV_R | ccgctcgagTCGGATGAAGAAAGAGGCCG | XhoI | |
| gtrX_F | cggggtaccAACCACTATCGGAAAGCGCA | KpnI | S. flexneri 3a |
| gtrX_R | ccgctcgagGGTTAACGCGCATGCTACTG | XhoI | |
| oacA_F | ataagaatgcggccgcAGAAACAGAAGCCACTGGAGC | NotI | S. flexneri 3a |
| oacA_R | cccaagcttCTGCGTGGAAAAGAACTCCAC | HindIII | |

TABLE 5
GLC-MS on purified Oag

| | | | | GMMA | | GMMA |
|---|---|---|---|---|---|---|
| | | GMMA 1a | GMMA 2a | mixed | GMMA 3a | hybrid |
| Linkage | RRT^{a} | scaffold | scaffold | 1 + 2 | natural | 1 + 3 |
| 2-Rha^{b} | 0.56 | 1.00 | 1.00 | 1.00 | 1.00 | 1.00 |
| 3-Rha^{b} | 0.57 | 0.50 | — | 0.22 | 0.86 | 0.68 |
| 2,3-Rha^{b} | 0.65 | — | — | — | 1.02 | 0.72 |
| 3,4-Rha^{b} | 0.64 | — | 0.64 | 0.37 | — | — |
| 3-GlcNAc | 1.10 | — | 1.02 | 0.31 | 1.32 | — |
| 3,4-GlcNAc | 1.15 | 0.77 | — | 0.29 | — | 0.80 |
| t-Glc | 0.61 | 0.74 | 0.74 | 0.71 | 1.53 | 1.97 |

^{a}Relative retention time;
^{b}the amount of Rha is underestimated because it is acid labile

TABLE 6
FACS typing analysis of all strains generated
by complementing serotype Y scaffold strain

| | Mean Fluorescence Intensity (FACS) | | | | | | |
|---|---|---|---|---|---|---|---|
| Strain | Type I | Type II | Type III | Type IV | Type V | Group 6 | Group 7(8) |
| Y | 25.6 | 26.7 | 29.7 | 33.2 | 33.7 | 29.4 | 24.6 |
| 1a | 9585 | 146 | 39.9 | 41.9 | 37.3 | 47.8 | 52.5 |
| 2a | 42.5 | 4748 | 38.6 | 40.6 | 42.5 | 38 | 50.7 |
| 4a | 31.5 | 30.5 | 27.6 | 4495 | 37.6 | 26.7 | 25.7 |
| 5a | 25 | 24.7 | 23.7 | 31.4 | 610 | 24.7 | 27.5 |
| X | 33.7 | 60.2 | 54.2 | 28.3 | 35.7 | 25.2 | 1574 |
| 3b | 24.5 | 27.1 | 860 | 28.2 | 33.7 | 1835 | 33.8 |
| 1a | 8211 | 104 | 30.4 | 27.2 | 23.4 | 39.9 | 34.5 |
| 2a | 25.4 | 3572 | 23.4 | 22.7 | 22.9 | 23.4 | 29.9 |
| 4a | 29.9 | 28.1 | 25.1 | 3903 | 30.8 | 24.2 | 24.3 |
| 5a | 22.9 | 24.5 | 22.5 | 29.3 | 330 | 23.8 | 27.1 |
| X | 26.3 | 60.2 | 54.2 | 26 | 28.8 | 25.2 | 1684 |
| 3b | 31.1 | 26.6 | 564 | 28 | 27.6 | 2148 | 35.3 |
| 1 + 2 | 5457 | 1449 | 27.4 | 27.2 | 23.8 | 27.8 | 31.5 |
| 4 + 5 | 29.5 | 29.9 | 24.7 | 3343 | 97.6 | 23.9 | 23.9 |
| 1 + 2 | 2085 | 2118 | 20.2 | 25.8 | 21.6 | 20.4 | 25.2 |
| 1 + 5 | 4661 | 70.3 | 25.6 | 23.7 | 106 | 27.6 | 40.4 |
| 1d | 5818 | 101 | 30.9 | 25.3 | 23.2 | 27.1 | 635 |
| 1b | 5928 | 75.8 | 39.8 | 27 | 26.3 | 3257 | 42.9 |
| 2 + 5 | 21.9 | 1108 | 21.1 | 19.2 | 164 | 19.7 | 22.5 |
| 2b | 37.1 | 3813 | 37.2 | 22.5 | 22.7 | 24 | 1809 |
| 2 + A | 23.8 | 876 | 686 | 23.8 | 26.6 | 1433 | 23 |
| 4 + 1 | 5476 | 84.6 | 26.4 | 1811 | 23.5 | 29.1 | 28.2 |
| 4 + 2 | 22.8 | 2344 | 21.4 | 1290 | 23 | 20.4 | 23.1 |
| 4 + 5 | 26.2 | 23.3 | 24.6 | 2965 | 161 | 25.5 | 26.2 |
| 4 + X | 44.1 | 55.2 | 42 | 2409 | 33.4 | 25.6 | 1420 |
| 4b | 27.4 | 28.4 | 30.9 | 4126 | 32.2 | 1429 | 25.5 |
| 5b | 23.3 | 27.1 | 28.2 | 24.4 | 1064 | 25.4 | 531 |
| 5 + A | 23.6 | 24.4 | 571 | 25.9 | 664 | 991 | 331 |
| 3a | 26 | 29.9 | 1786 | 26.6 | 29.8 | 739 | 687 |

TABLE 7
FACS typing analysis of all strains generated
by complementing S. flexneri 3a strain

| | Mean Fluorescence Intensity (FACS) | | | | | | |
|---|---|---|---|---|---|---|---|
| Strain | Type I | Type II | Type III | Type IV | Type V | Group 6 | Group 7(8) |
| 3a | 50.1 | 65.9 | 2960 | 57 | 55.9 | 1382 | 1460 |
| 3 + 1 | 9344 | 142 | 225 | 64.2 | 63.1 | 3427 | 925 |
| 3 + 2 | 50.2 | 2299 | 2379 | 49.6 | 53.8 | 885 | 2002 |
| 3 + 4 | 48 | 57.1 | 646 | 5834 | 52.8 | 2086 | 527 |
| 3 + 5 | 51.3 | 62.1 | 1730 | 56.3 | 849 | 898 | 1419 |
| 3 + 1 + 2 | 6632 | 1927 | 1013 | 61.8 | 58.2 | 3149 | 1915 |
| 3 + 4 + 5 | 47.7 | 60.3 | 762 | 5316 | 408 | 1818 | 831 |
| 3 + 1 + | 2958 | 411 | 84.9 | 4826 | 256 | 2795 | 666 |
| 2 + 4 + 5 | | | | | | | |

TABLE 8
Compatibility between OAg-modifying enzymes

| | gtrI | gtrII | gtrX | gtrIV | gtrV | oacA |
|---|---|---|---|---|---|---|
| gtrI | — | 0% hybrid | 100% hybrid | 0% hybrid | 100% hybrid | 100% hybrid |
| | | (steric hindrance) | (natural serotype) | (competition to | (unnatural serotype) | (natural serotype) |
| | | | | modify same residue) | | |
| gtrII | — | — | 100% hybrid | 70% hybrid | 60% hybrid | 0% hybrid |
| | | | (natural serotype) | (unnatural serotype) | (unnatural serotype) | (competition to |
| | | | | | | modify same residue) |
| gtrX | — | — | — | 45% hybrid | 100% hybrid | 100% hybrid |
| | | | | (unnatural serotype) | (natural serotype) | (natural serotype) |
| gtrIV | — | — | — | — | 40% hybrid | 100% hybrid |
| | | | | | (unnatural serotype) | (natural serotype) |
| gtrV | — | — | — | — | — | 0% hybrid |
| | | | | | | (steric hindrance) |
| oacA | — | — | — | — | — | — |

### EMBODIMENTS OF THE INVENTION

1. An outer membrane vesicle comprising at least one heterologous O-antigen.

2. A recombinant Shigella bacterium comprising at least one heterologous O-antigen.

3. The recombinant Shigella bacterium of embodiment 2, wherein the recombinant Shigella bacterium is a recombinant Shigella flexneri (S. flexneri) bacterium.

4. The outer membrane vesicle or recombinant Shigella bacterium of any one of the preceding embodiments, wherein the at least one heterologous O-antigen comprises O-antigen comprising epitopes from at least two different serotypes of Shigella.

5. The outer membrane vesicle or recombinant Shigella bacterium of embodiment 4, wherein the at least one heterologous O-antigen comprises O-antigen comprising epitopes from at least two different serotypes of S. flexneri.

6. The outer membrane vesicle or recombinant Shigella bacterium of embodiment 4 or 5, wherein the epitopes from at least two different serotypes of Shigella comprise glucosylation or O-acetylation patterns characteristic of at least two different serotypes of Shigella flexneri.

7. The outer membrane vesicle or recombinant Shigella bacterium of any one of embodiments 4 to 6, wherein the O-antigen comprising epitopes from at least two different serotypes of Shigella of S. flexneri comprises at least one hybrid O-antigen.

8. The outer membrane vesicle or recombinant Shigella bacterium of embodiment 7, wherein the at least one hybrid O-antigen comprises epitopes from at least two, or at least three different serotypes of Shigella.

9. The outer membrane vesicle or recombinant Shigella bacterium of embodiment 7 or 8, wherein the at least one hybrid O-antigen comprises epitopes from two, three or four different S. flexneri serotypes.

10. The outer membrane vesicle or recombinant Shigella bacterium of any one of embodiments 7 to 9, wherein the at least one hybrid O-antigen comprises epitopes from S. flexneri serotypes (i) 1a and 5a, (ii) 1a and X (i.e. serotype 1d), (iii) 1a and 3b (i.e. serotype 1b), (iv) 1a, 5a and X, (v) 1a, X and 3b, (vi) 2a and 4a, (vii) 2a and 5a, (viii) 2a and X (i.e. serotype 2b), (ix) 2a, 4a and 5a, (x) 2a, 4a and X, (xi) 2a, 5a and X, (xii) 2a, 4a, 5a and X, (xiii) 4a and 5a, (xiv) 4a and X, (xv) 4a and 3b (i.e. serotype 4b), (xvi) 4a, 5a and X, (xvii) 4a, 3b and X, (xviii) 5a and X (i.e. serotype 4b), or (xix) X and 3b (i.e. serotype 3a).

11. The outer membrane vesicle or recombinant Shigella bacterium of any one of embodiments 7 to 10, wherein the at least one hybrid O-antigen comprises epitopes from S. flexneri serotypes (i) 1a and 5a, (ii) 1a, 5a and X, (iii) 1a, 5a and 3b, (iv) 1a, X and 3b, (v) 2a and 4a, (vi) 2a and 5a, (vii) 2a, 4a and 5a, (viii) 2a, 4a and X, (ix) 2a, 5a and X, (x) 2a, 4a, 5a and X, (xi) 4a and 5a, (xii) 4a and X, (xiii) 4a, 5a and X, or (xiv) 4a, 3b and X.

12. The outer membrane vesicle or recombinant Shigella bacterium of any one of embodiments 7 to 11, wherein the at least one hybrid O-antigen comprises epitopes from S. flexneri serotypes (i) 1a and 5a, (ii) 1a and X, (iii) 1a and 3b, (iv) 2a and 4a, (v) 2a and 5a, (vi) 2a and X, (vii) 4a and 5a, (viii), 4a and X, (ix) 5a and X, (x) 4a and 3b, or (xi) 3b and X.

13. The outer membrane vesicle or recombinant Shigella bacterium of any one of embodiments 4 to 12, wherein the O-antigen comprising epitopes from at least two different strains of Shigella comprises a mixture of at least two different O-antigens.

14. The outer membrane vesicle or recombinant Shigella bacterium of embodiment 13, wherein the mixture of at least two different O-antigens comprises at least two different O-antigens from at least two different Shigella flexneri serotypes.

15. The outer membrane vesicle or recombinant Shigella bacterium of embodiment 13 or 14, wherein the mixture of at least two different O-antigens comprises epitopes from S. flexneri serotypes (i) 1a and 2a, (ii) 1a and 4a, (iii) 2a and 4a, (iv) 2a and 5a, (v) 2a and 3b, (vi) X and 4a, (vii) 4a and 5a, and/or (viii) 5a and 3b.

16. The outer membrane vesicle or recombinant Shigella bacterium of any one of embodiments 13 to 15, wherein the mixture of at least two different O-antigens comprises epitopes from S. flexneri serotypes (i) 1a and 2a, (ii) 1a and 4a, (iii) 2a and 3b, and/or (iv) 5a and 3a.

17. The outer membrane vesicle or recombinant Shigella bacterium of any one of embodiments 13 to 16, wherein the mixture of at least two different O-antigens comprises epitopes from S. flexneri serotypes 1a and 2a.

18. The outer membrane vesicle or recombinant Shigella bacterium of any one of embodiments 4 to 17, wherein the at least one heterologous O-antigen comprises at least one hybrid O-antigen and a mixture of at least two different O-antigens.

19. The outer membrane vesicle or recombinant Shigella bacterium of embodiment 18, wherein the at least one heterologous O-antigen comprises epitopes from S. flexneri serotypes (i) 2a and 4a, (ii) 2a and 5a, (iii) X and 4a, and/or (iv) 4a and 5a.

20. A method for preparing Shigella outer membrane vesicles comprising:
(i) providing at least one gene encoding an enzyme that modifies a Shigella O-antigen on at least one plasmid;
(ii) culturing a production Shigella bacterium in the presence of the at least one plasmid under conditions suitable for the enzyme to be expressed by the production Shigella bacterium and for Shigella outer membrane vesicles to be produced; and
(iii) harvesting the outer membrane vesicles.

21. A method for preparing a recombinant Shigella bacterium comprising at least one heterologous O-antigen comprising:
(i) providing at least one gene encoding an enzyme that modifies a Shigella O-antigen on at least one plasmid;
(ii) culturing a production Shigella bacterium in the presence of the at least one plasmid under conditions suitable for the enzyme to be expressed by the production Shigella bacterium to produce the recombinant Shigella bacterium comprising at least one heterologous O-antigen; and
(iii) harvesting the recombinant Shigella bacterium comprising at least one heterologous O-antigen.

22. A plasmid comprising at least one gene encoding an enzyme that modifies a Shigella O-antigen.

23. The plasmid or method of any one of embodiments 20 to 22, wherein the at least one gene comprises at least 1, at least 2, at least 3, between 1 and 5, or between 1 and 4 gene(s) encoding a glucosyltransferase.

24. The plasmid or method of embodiment 23, wherein the at least one gene encoding a glucosyltransferase comprises 1, 2, 3, 4 or 5 genes selected from the group consisting of gtrI, gtrII, gtrIV, gtrV and gtrX.

25. The plasmid or method any one of embodiments 20 to 24, wherein the at least one gene comprises at least 1, at least 2, or between 1 and 3 gene(s) encoding an O-antigen O-acetylase.

26. The plasmid or method of embodiment 25, wherein the at least one gene encoding an O-antigen O-acetylase comprises 1, 2 or 3 genes selected from the group consisting of oacA, oacB, and oacD.

27. The plasmid or method of embodiment 26, wherein the at least one gene encoding an O-antigen O-acetylase comprises an oacA gene.

28. The plasmid or method of any one of embodiments 20 to 27, wherein the at least one gene, or each at least one gene, is operably linked to a constitutive promoter.

29. The plasmid or method of any one of embodiments 20 to 28, wherein the at least one gene, or each at least one gene, is operably linked to its native promoter.

30. The method of any one of embodiments 20 or 23 to 29, wherein the method is a method for preparing Shigella outer membrane vesicles comprising O-antigen comprising epitopes from at least two, or at least three different serotypes of S. flexneri.

31. The method of any one of embodiments 21 or 23 to 29, wherein the method is a method for preparing a recombinant Shigella bacterium comprising O-antigen comprising epitopes from at least two, or at least three different serotypes of S. flexneri.

32. The method of any one of embodiments 20, 21, or 23 to 31, wherein the method is a method for preparing Shigella outer membrane vesicles or a recombinant Shigella bacterium according to any one of embodiments 1 to 20.

33. The method of any one of embodiments 20, 21, or 23 to 32, wherein the production Shigella bacterium is a recombinant Shigella flexneri bacterium.

34. The recombinant Shigella bacterium or method of any one of embodiments 2 to 20, 21, or 23 to 33, wherein the recombinant Shigella bacterium and/or the production Shigella bacterium comprises a genome encoding the enzymes required for expressing an S. flexneri O-antigen selected from the group consisting of serotype 1a, serotype 1d, serotype 2a, serotype 3a, serotype 3b, serotype 4a, serotype 4b, serotype 5a, and serotype X.

35. The recombinant Shigella bacterium or method of any one of embodiments 2 to 20, 21, or 23 to 34, wherein the recombinant Shigella bacterium and/or the production Shigella bacterium is a bacterium whose genome has been modified to delete one or more of the following genes that are present in the native genome: gtrI, gtrII, gtrIV, gtrV, gtrX, OacA, OacB and OacD and/or the recombinant Shigella bacterium and/or the production Shigella bacterium is an S. flexneri serotype Y bacterium.

36. The recombinant Shigella bacterium or method of any one of embodiments 2 to 20, 21, or 23 to 35, wherein the recombinant Shigella bacterium and/or the production Shigella bacterium is a bacterium whose genome has been modified to delete any of the following genes that are present in the native genome: gtrI, gtrII, gtrIV, gtrV, gtrX, oacA, oacB and OacD.

37. The recombinant Shigella bacterium or method of any one of embodiments 2 to 20, 21, or 23 to 36, wherein the recombinant Shigella bacterium and/or the production Shigella bacterium is a Shigella flexneri serotype 2a bacterium that has been modified by deleting the oacD, oacB and gtrII genes.

38. The method of any one of embodiments 20, 21 or 23 to 37, further comprising a step of preparing the production Shigella bacterium, prior to step (i), by modifying a Shigella bacterium to express a heterologous O-antigen.

39. The method of embodiment 38, wherein the step of preparing the production Shigella bacterium by modifying a Shigella bacterium to express a heterologous O-antigen comprises or consists of deleting one or more of the following genes that are present in the native genome: gtrI, gtrII, gtrIV, gtrV, gtrX, oacA, oacB and oacC.

40. The method of embodiment 38 or 39, wherein the step of preparing the production Shigella bacterium comprises or consists of deleting any of the following genes that are present in the native genome: gtrI, gtrII, gtrIV, gtrV, gtrX, oacA, oacB and OacD.

41. The method of any one of embodiments 38 to 40, wherein the step of preparing the production Shigella bacterium comprises or consists of modifying a Shigella flexneri serotype 2a bacteria by deleting the oacD, oacB and gtrII genes.

42. The method of any one of embodiments 20, 21 or 23 to 41, further comprising a step of purifying the outer membrane vesicles or the recombinant Shigella bacterium comprising at least one heterologous O-antigen.

43. The method of any one of embodiments 20, 21 or 23 to 42, further comprising a step of formulating the outer membrane vesicles or the recombinant Shigella bacterium comprising at least one heterologous O-antigen with additional components.

44. The method of embodiment 43, wherein the additional components comprise an adjuvant and a pharmaceutically acceptable excipient.

45. An outer membrane vesicle or recombinant Shigella bacterium obtained by the method of any one of embodiments 20, 21 or 23 to 44.

46. An outer membrane vesicle or recombinant Shigella bacterium obtainable by the method of any one of embodiments 20, 21 or 23 to 44.

47. The outer membrane vesicle, recombinant Shigella bacterium or method of any one of embodiments 1 to 21 or 23 to 46, wherein the outer membrane vesicle, production Shigella bacterium or recombinant Shigella bacterium comprises modified lipid A.

48. The outer membrane vesicle, recombinant Shigella bacterium or method of any one of embodiments 1 to 21, or 23 to 46, wherein the outer membrane vesicle, production Shigella bacterium or recombinant Shigella bacterium comprises modified lipid A that is less toxic compared to a corresponding wild-type lipid A.

49. The outer membrane vesicle, recombinant Shigella bacterium or method of embodiment 48, wherein the modified lipid A comprises a lipid A in which the C14 comprises a myristoyl group.

50. The outer membrane vesicle, recombinant Shigella bacterium or method of embodiment 48 or 49, wherein the modified lipid A comprises penta-acylated lipid A.

51. The recombinant Shigella bacterium or method of any one of embodiments 2 to 21, or 23 to 50, wherein the recombinant Shigella bacterium and/or the production Shigella bacterium has been modified by modification or deletion of the tolR gene, msbB gene and/or htrB gene.

52. An immunogenic composition comprising the outer membrane vesicle or recombinant Shigella bacterium of any one of embodiments 1 to 19, 34 to 37 or 45 to 51 and a pharmaceutically acceptable excipient.

53. A vaccine comprising the immunogenic composition of embodiment 52.

54. The immunogenic composition or vaccine of embodiment 52 or 53, which comprises an adjuvant.

55. The immunogenic composition or vaccine of embodiment 54, wherein the adjuvant is an aluminium adjuvant.

56. The immunogenic composition or vaccine of embodiment 55, wherein the aluminium adjuvant comprises aluminium hydroxide or aluminium phosphate.

57. The immunogenic composition or vaccine of any one of embodiments 52 to 56 for use in a method of preventing or treating Shigella infection.

58. A method for preventing or treating Shigella infection, comprising administering an effective amount of the immunogenic composition or vaccine of any one of embodiments 52 to 56 to a subject.

59. Use of the immunogenic composition or vaccine of any one of embodiments 52 to 56 in the manufacture of a medicament for use in a method of preventing or treating Shigella infection.

60. The immunogenic composition or vaccine for use, method of treatment or use of any one of embodiments 57 to 59, wherein the Shigella infection is a Shigella flexneri infection.

61. The immunogenic composition or vaccine for use or use of embodiment 57 or 59, wherein the method of preventing or treating Shigella infection comprises administering an effective amount of the immunogenic composition or vaccine of any one of embodiments 52 to 56 to a subject.

1. An outer membrane vesicle comprising at least one heterologous O-antigen.

2. A recombinant Shigella bacterium comprising at least one heterologous O-antigen, optionally wherein the recombinant Shigella bacterium is a recombinant Shigella flexneri (S. flexneri) bacterium.

3. The outer membrane vesicle or recombinant Shigella bacterium of any one of the preceding claims, wherein the at least one heterologous O-antigen comprises O-antigen comprising epitopes from at least two different serotypes of Shigella, optionally wherein the at least one heterologous O-antigen comprises O-antigen comprising epitopes from at least two different serotypes of S. flexneri.

4. The outer membrane vesicle or recombinant Shigella bacterium of any one of the preceding claims, wherein the O-antigen comprising epitopes from at least two different serotypes of Shigella of S. flexneri comprises at least one hybrid O-antigen.

5. The outer membrane vesicle or recombinant Shigella bacterium of claim 4, wherein the at least one hybrid O-antigen:
(a) comprises epitopes from at least two, or at least three different serotypes of Shigella;
(b) comprises epitopes from two, three or four different S. flexneri serotypes;
(c) comprises epitopes from S. flexneri serotypes (i) 1a and 5a, (ii) 1a and X (i.e. serotype 1d), (iii) 1a and 3b (i.e. serotype 1b), (iv) 1a, 5a and X, (v) 1a, X and 3b, (vi) 2a and 4a, (vii) 2a and 5a, (viii) 2a and X (i.e. serotype 2b), (ix) 2a, 4a and 5a, (x) 2a, 4a and X, (xi) 2a, 5a and X, (xii) 2a, 4a, 5a and X, (xiii) 4a and 5a, (xiv) 4a and X, (xv) 4a and 3b (i.e. serotype 4b), (xvi) 4a, 5a and X, (xvii) 4a, 3b and X, (xviii) 5a and X (i.e. serotype 4b), or (xix) X and 3b (i.e. serotype 3a); and/or
(d) comprises epitopes from S. flexneri serotypes (i) 1a and 5a, (ii) 1a, 5a and X, (iii) 1a, 5a and 3b, (iv) 1a, X and 3b, (v) 2a and 4a, (vi) 2a and 5a, (vii) 2a, 4a and 5a, (viii) 2a, 4a and X, (ix) 2a, 5a and X, (x) 2a, 4a, 5a and X, (xi) 4a and 5a, (xii) 4a and X, (xiii) 4a, 5a and X, or (xiv) 4a, 3b and X.

6. The outer membrane vesicle or recombinant Shigella bacterium of any one of claims 3 to 5, wherein the O-antigen comprising epitopes from at least two different strains of Shigella comprises a mixture of at least two different O-antigens.

7. The outer membrane vesicle or recombinant Shigella bacterium of claim 6, wherein the mixture of at least two different O-antigens:
(a) comprises at least two different O antigens from at least two different Shigella flexneri serotypes;
(b) comprises epitopes from S. flexneri serotypes (i) 1a and 2a, (ii) 1a and 4a, (iii) 2a and 4a, (iv) 2a and 5a, (v) 2a and 3b, (vi) X and 4a, (vii) 4a and 5a, and/or (viii) 5a and 3b;
(c) comprises epitopes from S. flexneri serotypes (i) 1a and 2a, (ii) 1a and 4a, (iii) 2a and 3b, and/or (iv) 5a and 3a; and/or
(d) comprises epitopes from S. flexneri serotypes 1a and 2a.

8. The outer membrane vesicle or recombinant Shigella bacterium of any one of claims 1 to 7, wherein the at least one heterologous O-antigen comprises at least one hybrid O-antigen and a mixture of at least two different O-antigens, optionally wherein the at least one heterologous O-antigen comprises epitopes from S. flexneri serotypes (i) 2a and 4a, (ii) 2a and 5a, (iii) X and 4a, and/or (iv) 4a and 5a.

9. A method for preparing Shigella outer membrane vesicles comprising:
(i) providing at least one gene encoding an enzyme that modifies a Shigella O-antigen on at least one plasmid;
(ii) culturing a production Shigella bacterium in the presence of the at least one plasmid under conditions suitable for the enzyme to be expressed by the production Shigella bacterium and for Shigella outer membrane vesicles to be produced; and
(iii) harvesting the outer membrane vesicles.

10. A method for preparing a recombinant Shigella bacterium comprising at least one heterologous O-antigen comprising:
(i) providing at least one gene encoding an enzyme that modifies a Shigella O-antigen on at least one plasmid;
(ii) culturing a production Shigella bacterium in the presence of the at least one plasmid under conditions suitable for the enzyme to be expressed by the production Shigella bacterium to produce the recombinant Shigella bacterium comprising at least one heterologous O-antigen; and
(iii) harvesting the recombinant Shigella bacterium comprising at least one heterologous O-antigen.

11. A plasmid comprising at least one gene encoding an enzyme that modifies a Shigella O-antigen.

12. The plasmid or method of any one of claims 9 to 11, wherein:
(i) the at least one gene comprises at least 1, at least 2, at least 3, between 1 and 5, or between 1 and 4 gene(s) encoding a glucosyltransferase;
(ii) the at least one gene comprises at least one gene encoding a glucosyltransferase which comprises 1, 2, 3, 4 or 5 genes selected from the group consisting of gtrI, gtrII, gtrIV, gtrV and gtrX;
(iii) the at least one gene comprises at least 1, at least 2, or between 1 and 3 gene(s) encoding an O-antigen O-acetylase;
(iv) the at least one gene comprises at least one gene encoding an O-antigen O-acetylase which comprises 1, 2 or 3 genes selected from the group consisting of oacA, oacB, and oacD;
(v) the at least one gene comprises at least one gene encoding an O-antigen O-acetylase which comprises an oacA gene;
(vi) the at least one gene, or each at least one gene, is operably linked to a constitutive promoter; and/or
(vii) the at least one gene, or each at least one gene, is operably linked to its native promoter.

13. The method of claim 9, 10 or 12, wherein:
(i) the method is a method for preparing Shigella outer membrane vesicles comprising O-antigen comprising epitopes from at least two, or at least three different serotypes of S. flexneri;
(ii) the method is a method for preparing a recombinant Shigella bacterium comprising O-antigen comprising epitopes from at least two, or at least three different serotypes of S. flexneri;
(iii) the method is a method for preparing Shigella outer membrane vesicles or a recombinant Shigella bacterium according to any one of claims 1 to 8; and/or
(iv) the production Shigella bacterium is a recombinant Shigella flexneri bacterium.

14. The recombinant Shigella bacterium or method of any one of claims 1 to 10 or 12 to 13, wherein:
(i) the recombinant Shigella bacterium and/or the production Shigella bacterium comprises a genome encoding the enzymes required for expressing an S. flexneri O-antigen selected from the group consisting of serotype 1a, serotype 1d, serotype 2a, serotype 3a, serotype 3b, serotype 4a, serotype 4b, serotype 5a, and serotype X;
(ii) the recombinant Shigella bacterium and/or the production Shigella bacterium is a bacterium whose genome has been modified to delete one or more of the following genes that are present in the native genome: gtrI, gtrII, gtrIV, gtrV, gtrX, oacA, oacB and oacD and/or the recombinant Shigella bacterium and/or the production Shigella bacterium is an S. flexneri serotype Y bacterium;
(iii) the recombinant Shigella bacterium and/or the production Shigella bacterium is a bacterium whose genome has been modified to delete any of the following genes that are present in the native genome: gtrI, gtrII, gtrIV, gtrV, gtrX, oacA, oacB and oacD; and/or
(iv) the recombinant Shigella bacterium and/or the production Shigella bacterium is a Shigella flexneri serotype 2a bacterium that has been modified by deleting the oacD, oacB and gtrII genes.

15. The method of any one of claims 9, 10, or 12 to 14, further comprising a step of preparing the production Shigella bacterium, prior to step (i), by modifying a Shigella bacterium to express a heterologous O-antigen, optionally wherein the step of preparing the production Shigella bacterium by modifying a Shigella bacterium to express a heterologous O-antigen comprises or consists of:
(i) deleting one or more of the following genes that are present in the native genome: gtrI, gtrII, gtrIV, gtrV, gtrX, oacA, oacB and oacC;
(ii) deleting any of the following genes that are present in the native genome: gtrI, gtrII, gtrIV, gtrV, gtrX, oacA, oacB and oacD; and/or
(iii) modifying a Shigella flexneri serotype 2a bacteria by deleting the oacD, oacB and gtrII genes.

16. The method of any one of claims 9, 10, or 12 to 15, further comprising a step of purifying the outer membrane vesicles or the recombinant Shigella bacterium comprising at least one heterologous O-antigen.

17. The method of any one of claims 9, 10, or 12 to 16, further comprising a step of formulating the outer membrane vesicles or the recombinant Shigella bacterium comprising at least one heterologous O-antigen with additional components, optionally wherein the additional components comprise an adjuvant and a pharmaceutically acceptable excipient.

18. An outer membrane vesicle or recombinant Shigella bacterium obtained by or obtainable by the method of any one of claim 9 or 12 to 17.

19. The outer membrane vesicle, recombinant Shigella bacterium or method of any one of claims 1 to 10 or 12 to 18, wherein:
(i) the outer membrane vesicle, production Shigella bacterium or recombinant Shigella bacterium comprises modified lipid A;
(ii) the outer membrane vesicle, production Shigella bacterium or recombinant Shigella bacterium comprises modified lipid A that is less toxic compared to a corresponding wild-type lipid A;
(iii) the outer membrane vesicle, production Shigella bacterium or recombinant Shigella bacterium comprises modified lipid A which comprises a lipid A in which the C14 comprises a myristoyl group;
(iv) the outer membrane vesicle, production Shigella bacterium or recombinant Shigella bacterium comprises modified lipid A which comprises penta-acylated lipid A; and/or
(v) the recombinant Shigella bacterium and/or the production Shigella bacterium has been modified by modification or deletion of the tolR gene, msbB gene and/or htrB gene.

20. An immunogenic composition comprising the outer membrane vesicle or recombinant Shigella bacterium of any one of claims 1 to 10, 14, or 18 to 19 and a pharmaceutically acceptable excipient.

21. A vaccine comprising the immunogenic composition of claim 20.

22. The immunogenic composition or vaccine of claim 20 or 21, which comprises:
(i) an adjuvant;
(ii) an aluminium adjuvant; and/or
(iii) an aluminium adjuvant which comprises aluminium hydroxide or aluminium phosphate.

23. The immunogenic composition or vaccine of any one of claims 20 to 22 for use in a method of preventing or treating Shigella infection, optionally wherein the Shigella infection is a Shigella flexneri infection.

24. A method for preventing or treating Shigella infection, comprising administering an effective amount of the immunogenic composition or vaccine of any one of claims 20 to 22 to a subject, optionally wherein the Shigella infection is a Shigella flexneri infection.

25. Use of the immunogenic composition or vaccine of any one of claims 20 to 22 in the manufacture of a medicament for use in a method preventing or treating Shigella infection, optionally wherein the Shigella infection is a Shigella flexneri infection.


SEQUENCE LISTING (32 sequences)

SEQ ID NO: 1 - DNA, 1521 bp, Shigella flexneri
  source 1..1521: mol_type = genomic DNA
atgagcatatgtattaaacaatcagcattaaaaatattattggctttatcagcactcctaataacttggttgactaccagatactttccagttgaaccggatgttgctaacagcccaattgtatggaggcatattttagagaatggtatatcatcaatccacgactggaaaccaaccgtggataattggtatttcaccgtttacccaattaactttttattttatatgctgttaggtgatgatggcttagtagcattaagattatcaactgcagttttctctattgctatagtaatagcagcgatgttaacgctacgtaaggcattcgggtttacccctgctttattttctattatacttctatctctaataccatatttttcatacacttatggttttgtatcacatcctttttctcataattcaacaaacgctttcggctttttatgcttgcttatatctgttttcaacatacagtataaaaatatatttatcactctcctcttaagtttgacagcattattttccagcgtatccgatccgtggtttacagctgcattttttattccactattaatatcttactttttattctctgtatgggataaaaaattatttaagcatactgcattaatactatttgcatgtttaatctctctttccaatgtattgcaaaatctacttaacatacctccccaccaatttgaaattgtatcactcaatgatatgattttaaatgcaaaatggtgcattctgttaataggaaaaagtcttaatttattagttgtcgacaataacgcaacatcttacgcctcatttgtcatttggttcattgccattattacatctgcgtggtttgttttatcggacaataaaaaaaacacataccgtatttatattgtgttattttcattattatctatcgcaggtattgtctcatcattcatactgagctataaatcccctgattatattagcatgcgtttctttatgaatgttacctgtttcgcactgatattgtgctgtatcggtacatcaacaaaagcaaagatattattttatctaatcgcatttttattttcgatcagttcaattaaatcttacacaaataatgcttcaccattacacgatcaggagaagattgttaaatcatacattgattttttaaaaaaaaataatctccattatgggtacggctctttttgggatttgtcaatgactgtaaattggttatctggtggggacatacagataactcctgttttctttaatgctgattcggggaaaattaactttactggcgtgagacaacagacactggcttcttggcatagcaaggaggcgttcaattctgctcctgagcgacagtttatagcagtttccatagctaacgaacctgaacgatgtaaagaaatgacatcctgccttgcaggtattcaagaacaattaggtaagcctgacgaagttttgaactttgaaggaagagtaatattagtatttaacaaaaaacttaacctatag

SEQ ID NO: 2 - DNA, 1461 bp, Shigella flexneri
  source 1..1461: mol_type = genomic DNA
atgattaaaattaatttatttaaaaatgcaaatctccttgccttcatatcatgttttgcaatatcaatttattgttattgggggtggttgtacgatggaacacttaatattgatggcgagtttacaaataatttttatcaaacaatcacgcttgggcggtggtttcacacttttttgcgacattactttctccctgagcctttttcactttatataacaccattaatagccttatcttttatcatcatttcagcatttataatctgcagatcgctaaagctagaatcttatgaattattgataggtatgttagtatttattaccttccctcagatctcctatcaattagagtttcttaaccaagctgatactgtgggaattgcttttctactggcagcgatatcagcaattatttttcactcgcaaaaaaataggattgtgatattttctggtatagtactgtccattctttcaatggcaatctaccaaacattcgtaacatatattattgcattcgtcattgggttgcagataaattcgataatacgaaatgagaaaaatattcgtgaatctttttatagttcatgtttatctctatccctcatagctttatctaccttaatttacctgctattaaccaaagctatcaagcattatttttcgcttgaatcgaacgagtacatctcaaattatatacaaaatgcaagcgatattaaatggcttgttaaatcagccatagataatatatataacttctataacaatcctcccactggtttaaacctatacaagtggttactgattcctttattaattctgatgtttaccctaacatataaattaaaaacaagatcaatttatttgatttcatcaatcattttcatttatatacttccggttatatttatcgttgttgttggctcaggggcgccacctcgcctgtttgttttaatgcctatagtagcagtaattttgttttcttgcttaagcaattttcgctctataaaatacctaaactgcatgttttttttatttattatatttaatggcgtttcaacatccaaaaatctatttttgaatgatactctcgcaagacagaaagatatctctttagctaaagaaatatcatacacatctcaaacaaaaggcatttcccttaacggaaaatatatatatatatatggttcaaacgactcaggaaatatgctctccatgagcgcagacacttttggaaaatcttttttttggtgggatggtggcaactattttaggatggttgcatttatgaattactatggaatctgtaattgcaaaccagcaaataaagaacaaatagagaagatttatccaattgtgaagagtttaccttcctggccgaatccagattcgatagctgaaataaatggtttggtgataataaaactatcagagaaaaaagggtggcttccatttaatatttag

SEQ ID NO: 3 - DNA, 1314 bp, Shigella flexneri
  source 1..1314: mol_type = genomic DNA
atggctgaaagaataaggtatgtattccttgcaattttttgtgcattactgtgcaaggagtcaattgacatatggatgatgaataatggcgacttcgatagggccataacacctttcatgaatgggatcagacagttctcacatgatggcacattactttataccctgaaggaaaatttcagttcaattgtgaactatgagtataaaagctcattcagttatattctttatttatacgcttatgttatatcgatattcacacacacatttgatcttaggttatggtcatctctttccaaaatcgtgtatatttgtgcgttatataaactatttgtagaaataaacaataaaaaatattcttatcttttatttccggtatcagcattattgctatttactccttccatactttcacaattcacagcattttatcaggatcaaatagtaatcactttacttccggtaattacttatataattcttaaaaaagaaatatcaaggtttgataaagtattactagttgcatgcgcagcgctaatcgccacatcaaaatctcagttcttctatattcccattttaatttctttaatttcaataatatttattgacataagagataaaaaatttcacttagctatggtaattgcgacgataattgcattgtcattttcatttttctcgccaagcgcaactaaaaataacagctatcactccctttatttcgggactttgctatacaataaaagcaatggaaaagaaaatccatcgtgggccgtggacgaatgcattggtgttgatgcatggggaaataaatttgatttagataagggagctgttaccacagaagatgctggagcctgcttcgcaaaaaataaagaaaggggattgaaggattctttgcttttgatgctaaagcaaccttccatgttcctccttcttccttttgattctggagttcgaacccaattaactgaagattacttccatgtcttcaaggaaaataagctaataataagtaagagtgtttttttgaatgaagttcaaaaaataaaagactctgctctatcaactgtgaggattcctgttgcatttatagccatgatagcatcactgatttttatccgaaaaaaatattctgttgcaatctttacattatctgcttttagcatctctcagttttatattagcttcattggcgaaggatatagagatttagacaagcatctttttgcaatgaacttttcatttgacctaatgatatttatagtactatcaattatattggataaggtagcatcaggaatttataagtaa

SEQ ID NO: 4 - DNA, 1254 bp, Shigella flexneri
  source 1..1254: mol_type = genomic DNA
atgaaaagccttaaaacatcatatgtaaaaaaactatttttcttaattgctgtatttttcattgcggttattttgatgtatttgcgccgtcctgatataatcagaacaccgcagttttgggcagaagatggtcatgtatggtatgctatggcatacaataatggcatattcacatcaatgatattccctcaaaatggatattatcaaaccatatcaaaacttattgcctccatatcattaaattttaatttaatgtatgcccctttaatttttaacatctccgcaattattgttagggcattgcttgtgtctttcatactctctggaagatttagttacgtaaacataacaccaagaatatttactgctttgtttattataataatgccggaagtatcagaagtgcacgccaatgttactaatgatcattggtatttatcactatatttacttcttgttatgttagctccaaagcctacaaataattatcaaaaagcacacgattatatagcgataatcatatgtggactaagtggtccatttatagtattcatggctccaatggtagggataaatatacttatacaaaaacgcttatctacaaaaaaaataaccactcacgaatacgttttcattgctatttgttttattcaatttatttctattatcatgacatcctctgagaccagagttgatatggctcttggagcaaatttcgcaacactttgcaaaatattaacaacaaaggtttttctgggtttatgggctaatggtgactttttatcacctttatggaatcatgattcaatttgcattgcaattacaatgatttgtctttccatcactatttttacatttattgtttccaattaccccatgcgctcggcaatagttttcgcaatactaacattaacgttttcactagcaaaaccaatgctttcgtctacaagtgagcaatggccattattaattcatggcggaggaagatattctgtcataccaacaattatatggtactctgtattggtttacttcttaaactatatttcccacttgcactttaaattcctaccatggatgttatatacaacatcgttaatctcatgtttatattttttcaatcttaccccactcccagattacgggtggaaagagcaagttaaaaaatttgaaagtttagcaccaggagaatcgtattctttcaagttcaaccctgcaggatggacgatgacattaataaaaaaacagccttaa

SEQ ID NO: 5 - DNA, 1266 bp, Shigella flexneri
  source 1..1266: mol_type = genomic DNA
atgaagatattcataatgcgcaattggcacaagatatcaatttttatacttgcatttactttgatttggcttaggcgcattgatattttgacaaatgcccagttttgggctgaggatgcagttttttggtataaagatgcatacgaacaaggttttttcagttcactcataaccccaagaaatggttattttcaaacagtatcaacactcattgttggtgcgacgacatttattaatccaatttacgcaccattattatcaaacttttttggaataataattagagctataatcatatggttcttatttaccgatagatttaaattcctaagcaccacatcaaaaatcttcatctctgtatatttaatttgcatgcctgggcttgatgaagtccaagccaatataacaaatgctcactggtatttatcattgtatgtagcaatgataataatatctgaagaatccaagtcaaaattatggaaagcacatgatttattttttatagtattatctggattgagcggtccatttattatatttataattgcatcatctgtttttaaatatttacacttatcaaaaagagtaataagcataagaggtttatacttattttatactcgactgccatatttggcaatgatcttgtgtgggctgatacaggcaacatcaataattcttacatttaatggaacaagatcacatgctccattaggattcagttttgatgttatgtcatctataatatcatctaatgtatttcttttttcatttgtcccttggaatatcgctgatgctggatgggataatcaccttttatcgtacacactatcaactttagtgattacattatgtgttttcctttatattaaaggaaattggcaaatgaaagtgtttgccacccttccaatacttattgtcgcattttctatggcaaaacctcaactggcagattctgttcctcaactgccaacacttgcaaccggaaacgggtcaaggtattttgtaaatattcatattgcaattttttcattaatatgcgtttgtttgtttcaatgcataaagaataaaaccttaaaaatattctttaaaatctatgtttcagtcatccttattgtaatgataaagttaaacttttttataacccctcttcctgacatgaactggtctcaaggagcagaattaataaataaagcaaaacatggagaagctgtctcatttgcagtgctccctccatggttaactcttgatttaataaaaaaataa

SEQ ID NO: 6 - DNA, 1002 bp, Shigella flexneri
  source 1..1002: mol_type = genomic DNA
atgcataagagcaactgctttgatacggcaaggcttgtggcagctatgatggttctaatcagtcaccactacgcgctctctggacaactcgagccgtatatattcggctttgaaagtgctggcggcattgctgttatcatattcttttccatatcaggatacctgatttcaaaatcagcaatcagaagtgattcctttattgatttcatggctaaaagagcaagaagaatattccctgctcttgtcccttgctctgtactgacgtattttttatttgggtggatgcttaacaacttcagtacagaatattttagtcatgacattatccggaagaccataagctctattttaatgagtcaggctccagatgccgacattacatcacatttaattcatcccggtattaacggcagcttgtggacactaccactggagtttatgtgttacataataactggagtggctgttgctctactaaagagcggaaaaacgttcatagttatactattgatttttgtttcactttcattagttgaaagtgtatctgagaacaaggatgtaatattctccattccagtatggctgtatcctttgcgggggatggctttcttttttggtgccactatggcgatgtatgagaaatcttggaatgtaagtaatgtgaaaatttccgtgatttctttgcttgctatgtacgcatacgcgagctatggcaaaggcattgactatacaatggcttgctatattctggtttctttctcaacaatagctatatgcacatcctttggagatccactggttaagggtaggtttgattactcttatggtgtatacatttatgcattccccgttcagcaggttattataaatacattgcatattggcttttacccttcgatgttgttaagtgccattgtcgttcttttcttggctcatctttcttggaatctaattgaaaaaagattccttacacgaagctcgccaaaattatccctgaattga

SEQ ID NO: 7 - DNA, 1173 bp, Shigella flexneri
  source 1..1173: mol_type = genomic DNA
atgcatatgattgaaattaacagtttattattaataacatccgtgatattgatgtcgttattagctgtggggttatttgataaaatttccccaataaatcttgttgagcacggcaggaataatcaaatagatggaatgcgggggttcttagcaattttcgtgcttattcatcacgcagcaatttggaatggatacttgtcatctggagtatgggaagcaccttcatcaaatctgttagcaaatttaggccaagttggtgtgtcattcttttttatgattactggttatctgttcttttcaaagattatctctggagatcaggactggacaaggctttatgtatcaagattactacgattaaccccaatgttcatagttagcctatgtctcatttttatcattgtaggttttaagtctggatggagaatgcaggtatccacagaagagctttttgtgtcaataatgaagtggttgccattcactgcgctaggtatgccgaacattaatgacgtaaaagattcatttactatcaatgccgctgtaacatggacacttgtatatgaatggttcttttatttttctcttccggtaatttccgcgctcataaaaagaaaagtcagtatttacatggttatgattagcgcaatatcgctatttgttttcattttatttttcagtaaaatacacatagcatcatttctttttgggctcctggcatttttgctaaataaatcaaagatagtcaatggaattgccaaagcaaaagtaacaccaattataataactgcgataatggtttttgaaatgacgtatttcaaaacaacttacgcaccgctaccactgattctttgtggaataacatttatcattatcgcatcaggttgtgatttgtatggaatattaagattaaatataaccagaaagctaggagaaactacctacagcgtttaccttctgcatgggatattcctttattgcctaatgacgtggattattcctaataattacactgaaaatacatttatcatactggtaagcacaactgcatttcttattacgttcacatcatgcctaacttttaaattaatagagacgccattcatcaaattaacaaaacaaaccacaactttagtaaaagaattaatacccacattaacaaacaacaatcaataa

SEQ ID NO: 8 - DNA, 1050 bp, Shigella flexneri
  source 1..1050: mol_type = genomic DNA
atgtataataacaaaatcgacagcaacagaaaaataagacatgattgggttgatgctttaaaatttctcggtatttttgctatatatttaggtcaccttgggttgggggctggcaaattgtatccttttgtttttagttatcacgtcccactattctttttcgctgctggtttttttactatcaaaaaaaatgacttatctgttttcgattatatcaagtcaaagttttacagattaatgattccatatttcacttttgcattttctatattaataataaacacaattaactcaggtgagacgattgattatatatacaaccacatttatgatataatatatggtgttagaaataaccaattcgtaggaacaatatggtttataaactgcctttttgttataatagcaattgatgcaatctttagagaaatagtcaagaacaatatagtgattttaatcatatcactgttatcgttcatgttgtcacaaacagtgctcaatcataacccattactagatcctcaatggttttggaacatagactccgctatggcatactggtggttattgcctctcggtagatgtatgtttttagaattaacacgtgacaggttttttggaaaatctaaaattggtttcattgtcttctcaattaccgctataatgagtgcgtatcaactacttaaccagaaacctttactttttaagattatctcaatattcaatgcagacataatatcaagtagttatattcaagctattaatactattatcaccacggtaggcttgataatctttaatattttcatagcaaaaataatttgtggaaatgactttattgtaagagctggaagaaatactcttaacatatgtggaatggagtatatcaccaaattatttatccctatggctttagcaattataggcttcagcgtgactattccaaatcctatctgcgcaatcatatatacctgtatttgtgtatatgtatcagataaaattggccattggctatcgaggacagtcggtgggccatttttgataaaatga

SEQ ID NO: 9 - DNA, 29 bp, Shigella flexneri
  source 1..29: mol_type = genomic DNA
tcgatcaattgagacttggatgatagact

SEQ ID NO: 10 - DNA, 29 bp, Shigella flexneri
  source 1..29: mol_type = genomic DNA
tcgatcaattgagacttggatgatagact

SEQ ID NO: 11 - DNA, 29 bp, Shigella flexneri
  source 1..29: mol_type = genomic DNA
tcgatcaattgagacttggatgatagact

SEQ ID NO: 12 - DNA, 29 bp, Shigella flexneri
  source 1..29: mol_type = genomic DNA
tcgatcaattgagacttggatgatagact

SEQ ID NO: 13 - DNA, 29 bp, Shigella flexneri
  source 1..29: mol_type = genomic DNA
tcgatcaattgagacttggatgatagact

SEQ ID NO: 14 - DNA, 29 bp, Shigella flexneri
  source 1..29: mol_type = genomic DNA
tggactatgacataaagatctgatatttt

SEQ ID NO: 15 - DNA, 31 bp, Shigella flexneri
  source 1..31: mol_type = genomic DNA
ttgtttcatcagttacacataactatatgat

SEQ ID NO: 16 - DNA, 29 bp, Shigella flexneri
  source 1..29: mol_type = genomic DNA
ttgaactcgttgattaataatcataaact

SEQ ID NO: 17 - DNA, 70 bp, synthetic construct
  source 1..70: mol_type = other DNA
accgccaggcgtttaccgttagcgagagcaacaaggggtaagccatggccgtgtaggctggagctgcttc

SEQ ID NO: 18 - DNA, 70 bp, synthetic construct
  source 1..70: mol_type = other DNA
acccgctctctttcaagcaagggaaacgcagatgtttagataggctgcgtcatatgaatatcctccttag

SEQ ID NO: 19 - DNA, 70 bp, synthetic construct
  source 1..70: mol_type = other DNA
ttttatctgatacatatacacaaatacaggtatatatgattgcgcagatagtcttgagcgattgtgtagg

SEQ ID NO: 20 - DNA, 70 bp, synthetic construct
  source 1..70: mol_type = other DNA
tggtttgttttgttaatttgatgaatggcgtctctattaatttaaaagtttcctccttagttcctattcc

SEQ ID NO: 21 - DNA, 29 bp, synthetic construct
  source 1..29: mol_type = other DNA
cgcggatccaagccttgcaagccattgtg

SEQ ID NO: 22 - DNA, 30 bp, synthetic construct
  source 1..30: mol_type = other DNA
catgccatggcccgtcgaaaaagacgggtt

SEQ ID NO: 23 - DNA, 36 bp, synthetic construct
  source 1..36: mol_type = other DNA
ataagaatgcggccgcaagccttgcaagccattgtg

SEQ ID NO: 24 - DNA, 29 bp, synthetic construct
  source 1..29: mol_type = other DNA
cccaagcttgagcttgaaaaagggaggcg

SEQ ID NO: 25 - DNA, 37 bp, synthetic construct
  source 1..37: mol_type = other DNA
ataagaatgcggccgctgtcacaaatacggcacaacg

SEQ ID NO: 26 - DNA, 34 bp, synthetic construct
  source 1..34: mol_type = other DNA
cccaagcttagagcaagatttaacgctacataaa

SEQ ID NO: 27 - DNA, 29 bp, synthetic construct
  source 1..29: mol_type = other DNA
cggggtaccacacgtcccaccacatcaaa

SEQ ID NO: 28 - DNA, 29 bp, synthetic construct
  source 1..29: mol_type = other DNA
ccgctcgagtcggatgaagaaagaggccg

SEQ ID NO: 29 - DNA, 29 bp, synthetic construct
  source 1..29: mol_type = other DNA
cggggtaccaaccactatcggaaagcgca

SEQ ID NO: 30 - DNA, 29 bp, synthetic construct
  source 1..29: mol_type = other DNA
ccgctcgagggttaacgcgcatgctactg

SEQ ID NO: 31 - DNA, 37 bp, synthetic construct
  source 1..37: mol_type = other DNA
ataagaatgcggccgcagaaacagaagccactggagc

SEQ ID NO: 32 - DNA, 30 bp, synthetic construct
  source 1..30: mol_type = other DNA
cccaagcttctgcgtggaaaagaactccac